(19) United States
(12) Patent Application Publication
(10) Pub. No.: US 2026/0033722 A1
(43) Pub. Date: Feb. 5, 2026

(54) SYSTEMS AND METHODS FOR MAGNETIC RESONANCE IMAGING

(71) Applicant: SHANGHAI UNITED IMAGING HEALTHCARE CO., LTD., Shanghai (CN)

(72) Inventors: Qi LIU, Houston, TX (US); Jingyuan LYU, Houston, TX (US); Jian XU, Houston, TX (US)

(73) Assignee: SHANGHAI UNITED IMAGING HEALTHCARE CO., LTD., Shanghai (CN)

(21) Appl. No.: 18/732,580

(22) Filed: Jun. 3, 2024

Related U.S. Application Data
(63) Continuation-in-part of application No. 17/822,436, filed on Aug. 26, 2022, now Pat. No. 12,000,919.

(30) Foreign Application Priority Data
Jan. 5, 2024 (CN) 202410017386.0

(51) Int. Cl. A61B 5/00; A61B 5/055; A61N 5/10; G01R 33/56; G01R 33/563

(52) U.S. Cl. CPC A61B 5/0036; A61B 5/055; A61N 5/1067; G01R 33/5608; G01R 33/563; A61N 2005/1055

(57) ABSTRACT

A system and method for MRI-guided radiotherapy may be provided. The method may include obtaining first auxiliary signals and second auxiliary signals collected by an MRI device during an MRI scan of a target subject, wherein the first auxiliary signals are collected by the MRI device at a first time before radiotherapy rays are emitted, and the second auxiliary signals are collected by the MRI device at a second time after the radiotherapy rays are emitted. The method may also include determining whether a first motion amplitude of the target subject from the first time to the second time is greater than a threshold based on the first auxiliary signals and the second auxiliary signals. The method may further include determining whether to stop emitting the radiotherapy rays based on a determination result of whether the first motion amplitude is greater than the threshold.

### CROSS-REFERENCE TO RELATED APPLICATIONS

This application is a continuation-in-part of U.S. patent application Ser. No. 17/822,436, filed on Aug. 26, 2022, and claims priority of Chinese Patent Application No. 202410017386.0, filed on Jan. 5, 2024, the contents of each of which are hereby incorporated by reference.

### TECHNICAL FIELD

The present disclosure relates to medical imaging, and in particular, to systems and methods for magnetic resonance imaging (MRI).

### BACKGROUND

MRI is an important clinical tool for disease diagnosis and/or treatment. For example, in radiation therapy, a motion tracking technique needs to be used to improve the precision of the radiation delivery to the target in the presence of a physiological motion of the target and/or an organ-at-risk (OAR) near the target. Recently, MRI technology has been used in radiation therapy to provide accurate images for tracking the target and/or the ORA. However, conventional MRI techniques have a long latency, which results in that the radiation therapy cannot be timely adjusted to adapt to the motion of the target and/or the OAR. Therefore, it is desirable to provide systems and methods for real-time MRI.

MRI-guided radiotherapy has great clinical value in whole-body tumor radiotherapy, especially in soft tissue tumor radiotherapy. The integrated MRI-guided radiotherapy device can detect a target region (also referred to as a region of interest (ROI)) that needs to receive radiotherapy in real time through MRI during treatment to distinguish tumors from normal tissues, thereby greatly improving the accuracy of radiotherapy. Real-time MRI plays a critical role in the MRI-guided radiotherapy device. Real-time imaging can capture the change in positions of tumor tissues in time and dynamically adjust the radiotherapy plan, thereby achieving accurate radiotherapy for tissues.

Therefore, it is desirable to provide efficient and accurate systems and methods for MRI-guided radiotherapy.

### SUMMARY

According to an aspect of the present disclosure, a system for MRI may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may determine an initial spatial factor U₀ based on first target MRI signals collected in a first imaging stage of an MRI scan of a subject. The system may also obtain second target MRI signals {M₁, M₂, . . . , Mₙ}. Mᵢ may be collect at time Tᵢ among time series {T₁, T₂, . . . , Tₙ} during a second imaging stage of the MRI scan after the first imaging stage. The system may also determine temporal factors {φ₁, φ₂, . . . , φₙ} and spatial factors {U₁, U₂, . . . , Uₘ}, m being smaller than or equal to n, di being determined based on the second target MRI signals Mᵢ, Uⱼ being determined based on the second target MRI signals {M₁, M₂, . . . , Mt}, 1<t<m and Mt being the latest second target MRI signals obtained before the determination of Uⱼ. The system may further generate real-time MRI images {A₁, A₂, . . . , Aₙ}, Aᵢ reflecting the status of the subject at the time Tᵢ, and being generated based on the temporal factor φᵢ and one of the initial spatial factor U₀ or the latest spatial factor determined before the time Tᵢ.

In some embodiments, the obtaining of the second target MRI signals {M₁, M₂, . . . , Mₙ}, the determination of the temporal factors {φ₁, φ₂, . . . , φₙ}, and the generation of the real-time MRI images {A₁, A₂, . . . , Aₙ} may be implemented by a first thread. The determination of the spatial factors {U₁, U₂, . . . , Uₘ} may be implemented by a second thread. The second thread may be further configured to feed the determined spatial factors {U₁, U₂, . . . , Uₘ} to the first thread.

In some embodiments, the first thread may be further configured to determine second temporal factor {φ′₁, φ′₂, . . . , φ′ₘ}, φ′ⱼ being determined based on the second target MRI signals {M₁, M₂, . . . , Mₜ}, and feed the determined second temporal factors {φ′₁, φ′₂, . . . , φ′ₘ} to the second thread. The spatial factor U; may be determined by the second thread based on the second target MRI signals {M₁, M₂, . . . , Mₜ}, and the second temporal factor φ′ⱼ.

In some embodiments, the first target MRI signals may include first target auxiliary signals and first target imaging signals. To determine an initial spatial factor U₀ based on first target MRI signals collected in a first imaging stage of an MRI scan of a subject, the system may determine an initial temporal factor φ₀ based on the first target auxiliary signals. The system may further determine the initial spatial factor U₀ based on the initial temporal factor φ₀ and the first target imaging signals.

In some embodiments, the at least one processor may be further configured to direct the system to perform one or more of the following operations. The system may determine a transformation coefficient T based on the first target auxiliary signals. The transformation coefficient may represent a relationship between auxiliary signals and temporal factors. The second target MRI signals Mᵢ may comprise a plurality of second target auxiliary signals. The temporal factor φᵢ may be determined based on the transformation coefficient T and the second target auxiliary signals of the second target MRI signals Mᵢ.

In some embodiments, to determine the temporal factor φᵢ, the system may update the value of the transformation coefficient T based on the plurality of second target auxiliary signals. The system may also determine the temporal factor φᵢ based on the second target auxiliary signals of the second target MRI signals Mᵢ and the updated transformation coefficient.

In some embodiments, the at least one processor may be further configured to direct the system to perform one or more of the following operations. The system may monitor body motion of the subject during the second imaging stage. In response to detecting that a magnitude of the monitored body motion of the subject exceeds a magnitude threshold, the system may determine a next spatial factor among the spatial factors {U₁, U₂, . . . , Uₘ}.

In some embodiments, to determine the spatial factor Uⱼ, the system may determine a second temporal factor φ′ⱼ based on the second target MRI signals {M₁, M₂, . . . , Mₜ}. The system may determine a reference spatial factor based on the second temporal factor φ′ⱼ and the second target MRI signals {Mₜ₋₁, . . . , Mₜ}, Mₜ₋₁ being the latest second target MRI signals obtained before the determination of Uⱼ₋₁. The system may further determine the spatial factor U based on the spatial factor Uⱼ₋₁ and the reference spatial factor.

In some embodiments, to determine the spatial factor Uⱼ, the system may determine a second temporal factor φ′ⱼ based on the second target MRI signals {M₁, M₂, . . . , Mₜ}. The system may also update coil sensitivity maps of coils based on the second target MRI signals {M₁, M₂, . . . , Mₜ}. The system may determine the spatial factor Uⱼ based on the second temporal factor φ′ⱼ the second target MRI signals {M₁, M₂, . . . , Mₜ}, and the updated coil sensitivity maps.

In some embodiments, the real-time MRI image of the subject may be a three-dimension (3D) image.

In some embodiments, the temporal factor q; may be determined based on the second target MRI signals M& Within a time period of 50 milliseconds.

In some embodiments, the spatial factor U; may be determined based on second target MRI signals {M₁, M₂, . . . , Mₜ} within a time period of 500 milliseconds.

According to another aspect of the present disclosure, a system for MRI may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may determine an initial spatial factor based on first target MRI signals collected in a first imaging stage of an MRI scan of a subject. The system may also obtain second target MRI signals collected during a second imaging stage of the MRI scan after the first imaging stage. The system may also determine temporal factors or/and updated spatial factors. Each of the temporal factors may be determined based on the second target MRI signals collected before the determination of the temporal factor. Each of the updated spatial factors may be determined based on the second target MRI signals collected before the determination of the updated spatial factor. The system may further generate real-time MRI images within the second imaging stage. Each of the real-time MRI images may be generated based on the latest determined temporal factor and one of the initial spatial factor or the latest determined spatial factor.

According to yet another aspect of the present disclosure, a method for MRI may be provided. The method may include determining an initial spatial factor U₀ Based on first target MRI signals collected in a first imaging stage of an MRI scan of a subject. The method may also include obtaining second target MRI signals {M₁, M₂, . . . , Mₙ}, Mᵢ being collect at time Tᵢ among time series {T₁, T₂, . . . , Tₙ} during a second imaging stage of the MRI scan after the first imaging stage. The method may also include determining temporal factors {φ₁, φ₂, . . . , φₙ} and spatial factors {U₁, U₂, . . . , Uₘ}, m being smaller than or equal to n, q; Being determined based on the second target MRI signals Mᵢ, Uⱼ being determined based on the second target MRI signals {M₁, M₂, . . . , Mₜ}, 1<t<m and Mₜ being the latest second target MRI signals obtained before the determination of Uⱼ. The method may further include generating real-time MRI images {A₁, A₂, . . . , Aₙ}, Aᵢ reflecting the status of the subject at the time Tᵢ, and being generated based on the temporal factor φᵢ and one of the initial spatial factor U₀ or the latest spatial factor determined before the time Tᵢ.

According to yet another aspect of the present disclosure, a method for MRI may be provided. The method may include determining an initial spatial factor based on first target MRI signals collected in a first imaging stage of an MRI scan of a subject. The method may also include obtaining second target MRI signals collected during a second imaging stage of the MRI scan after the first imaging stage. The method may also include determining temporal factors or/and updated spatial factors. Each of the temporal factors may be determined based on the second target MRI signals collected before the determination of the temporal factor. Each of the updated spatial factors may be determined based on the second target MRI signals collected before the determination of the updated spatial factor. The method may further include generating real-time MRI images within the second imaging stage. Each of the real-time MRI images may be generated based on the latest determined temporal factor and one of the initial spatial factor or the latest determined spatial factor.

According to yet another aspect of the present disclosure, a non-transitory computer readable medium may be provided. The non-transitory computer readable medium may comprise at least one set of instructions for MRI. When executed by one or more processors of a computing device, the at least one set of instructions causes the computing device to perform a method. The method may include determining an initial spatial factor U₀ Based on first target MRI signals collected in a first imaging stage of an MRI scan of a subject. The method may also include obtaining second target MRI signals {M₁, M₂, . . . , Mₙ}, Mᵢ being collect at time Tᵢ Among time series {T₁, T₂, . . . , Tₙ} during a second imaging stage of the MRI scan after the first imaging stage. The method may also include determining temporal factors {φ₁, φ₂, . . . , φₙ} and spatial factors {U₁, U₂, . . . , Uₘ}, m being smaller than or equal to n, φᵢ being determined based on the second target MRI signals Mᵢ, Uⱼ being determined based on the second target MRI signals {M₁, M₂, . . . , Mₜ}, 1<t<m and Mₜ being the latest second target MRI signals obtained before the determination of Uⱼ. The method may further include generating real-time MRI images {A₁, A₂, . . . , Aₙ}, Aᵢ reflecting the status of the subject at the time Tᵢ, and being generated based on the temporal factor φᵢ and one of the initial spatial factor U₀ or the latest spatial factor determined before the time Tᵢ.

According to yet another aspect of the present disclosure, a non-transitory computer readable medium may be provided. The non-transitory computer readable medium may comprise at least one set of instructions for MRI. When executed by one or more processors of a computing device, the at least one set of instructions causes the computing device to perform a method. The method may include determining an initial spatial factor based on first target MRI signals collected in a first imaging stage of an MRI scan of a subject. The method may also include obtaining second target MRI signals collected during a second imaging stage of the MRI scan after the first imaging stage. The method may also include determining temporal factors or/and updated spatial factors. Each of the temporal factors may be determined based on the second target MRI signals collected before the determination of the temporal factor. Each of the updated spatial factors may be determined based on the second target MRI signals collected before the determination of the updated spatial factor. The method may further include generating real-time MRI images within the second imaging stage. Each of the real-time MRI images may be generated based on the latest determined temporal factor and one of the initial spatial factor or the latest determined spatial factor.

According to yet another aspect of the present disclosure, a method for MRI-guided radiotherapy may be provided. The method may include obtaining first auxiliary signals and second auxiliary signals collected by an MRI device during an MRI scan of a target subject, wherein the first auxiliary signals are collected by the MRI device at a first time before radiotherapy rays are emitted, and the second auxiliary signals are collected by the MRI device at a second time after the radiotherapy rays are emitted. The method may also include determining whether a first motion amplitude of the target subject from the first time to the second time is greater than a threshold based on the first auxiliary signals and the second auxiliary signals. The method may further include determining whether to stop emitting the radiotherapy rays based on a determination result of whether the first motion amplitude is greater than the threshold.

In some embodiments, the method may further include the following operations. The method may include obtaining first imaging signals collected by the MRI device before the radiotherapy rays are emitted. The method may also include determining an initial temporal factor and an initial spatial factor based on the first auxiliary signals and the first imaging signals. In response to determining that the first motion amplitude is smaller than or equal to the threshold, the method may include determining a first updated temporal factor based on the second auxiliary signals. Further, the method may include generating a first MRI image of the target subject, wherein the first MRI image corresponds to the second time based on the first updated temporal factor and the initial spatial factor.

In some embodiments, to determine the initial temporal factor, the method may further include the following operations. The method may include obtaining a transformation coefficient, the transformation coefficient representing a relationship between auxiliary signals and temporal factors. The method may further include determining the initial temporal factor based on the transformation coefficient and the first auxiliary signals.

In some embodiments, the initial temporal factor may relate to at least one time-varying dimension of the target subject, the initial spatial factor may reflect a relationship between pixel information of the target subject in the image domain and spatial information of the target subject in the physical domain.

In some embodiments, the first MRI image of the target subject may be a three-dimension (3D) image.

In some embodiments, the method may further include the following operations. In response to determining that the first motion amplitude is greater than the threshold, the method may include instructing a radiotherapy device to stop emitting the radiotherapy rays. The method may also include obtaining third auxiliary signals collected by the MRI device at a third time, wherein the third time is later than the second time. The method may include determining whether a second motion amplitude of the target subject from the first time to the third time is greater than the threshold based on the first auxiliary signals and the third auxiliary signals. Further, in response to determining that the second motion amplitude is smaller than or equal to the threshold, the method may include instructing the radiotherapy device to continue emitting the radiotherapy rays.

In some embodiments, the method may further include the following operations. The method may include obtaining first imaging signals collected by the MRI device before the radiotherapy rays are emitted. The method may include determining an initial temporal factor and an initial spatial factor based on the first auxiliary signals and the first imaging signals. In response to determining that the second motion amplitude is smaller than or equal to the threshold, the method may also include determining a second updated temporal factor based on the third auxiliary signals. Further, the method may include generating a second MRI image of the target subject, wherein the second MRI image corresponds to the third time based on the second updated temporal factor and the initial spatial factor.

In some embodiments, in response to determining that the second motion amplitude is greater than the threshold, the method may further include the following operations. The method may include determining whether a third motion amplitude of the target subject from the second time to the third time is greater than the threshold based on the second auxiliary signals and the third auxiliary signals. In response to determining that the third motion amplitude is smaller than or equal to the threshold, the method may include instructing the radiotherapy device to continue emitting the radiotherapy rays.

In some embodiments, in response to determining that the third motion amplitude is smaller than or equal to the threshold, the method may further include the following operations. The method may include obtaining first imaging signals collected by the MRI device before the radiotherapy rays are emitted. The method may also include determining an initial temporal factor and an initial spatial factor based on the first auxiliary signals and the first imaging signals. The method may include determining a second updated temporal factor based on the third auxiliary signals. The method may also include determining an updated spatial factor based on second imaging signals collected by the MRI device between the second time and the third time and the second updated temporal factor. Further, the method may include generating, based on the second updated temporal factor and the updated spatial factor, a third MRI image of the target subject, wherein the third MRI image corresponds to the third time.

In some embodiments, to determine an updated spatial factor, based on second imaging signals collected by the MRI device between the second time and the third time and the second updated temporal factor, the method may further include the following operations. The method may include determining a reference spatial factor based on the second updated temporal factor and the second imaging signals. The method may include determining the updated spatial factor based on the initial spatial factor and the reference spatial factor.

In some embodiments, to determine an updated spatial factor based on second imaging signals collected by the MRI device between the second time and the third time and the second updated temporal factor, the method may further include the following operations. The method may include updating coil sensitivity maps of coils based on the second imaging signals. The method may further include determining the updated spatial factor based on the second updated temporal factor, the second imaging signals, and the updated coil sensitivity maps.

In some embodiments, the second updated temporal factor may be determined based on the third auxiliary signals within a time period of 50 milliseconds.

In some embodiments, the updated spatial factor may be determined based on the second imaging signals within a time period of 500 milliseconds.

In some embodiments, in response to determining that the third motion amplitude is smaller than the threshold, to instruct the radiotherapy device to continue emitting the radiotherapy rays, the method may further include the following operations. The method may include determining position information of a region of interest (ROI) of the target subject at the third time based on the third MRI image. The method may also include updating radiation parameters of the radiotherapy rays based on the position information. The method may further include instructing the radiotherapy device to continue emitting the radiotherapy rays based on the updated radiation parameters.

In some embodiments, the first auxiliary signal and the second auxiliary signals may have a first readout direction. To determine whether a first motion amplitude of the target subject from the first time to the second time is greater than a threshold based on the first auxiliary signals and the second auxiliary signals, the method may further include the following operations. The method may include obtaining first reference auxiliary signals and second reference auxiliary signals collected by the MRI device, wherein the first reference auxiliary signals are collected after the first time, and the second reference auxiliary signals are collected after the second time, the first reference auxiliary signals and the second reference auxiliary signals have a second readout direction, and the second readout direction is different from the first readout direction. Further, the method may include determining whether the first motion amplitude of the target subject from the first time to the second time is greater than the threshold based on the first auxiliary signals, the second auxiliary signals, the first reference auxiliary signals, and the second reference auxiliary signals.

According to an aspect of the present disclosure, a system for MRI-guided radiotherapy may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may obtain first auxiliary signals and second auxiliary signals collected by an MRI device during an MRI scan of a target subject, wherein the first auxiliary signals are collected by the MRI device at a first time before radiotherapy rays are emitted, and the second auxiliary signals are collected by the MRI device at a second time after the radiotherapy rays are emitted. The system may also determine whether a first motion amplitude of the target subject from the first time to the second time is greater than a threshold based on the first auxiliary signals and the second auxiliary signals. The system may further determine whether to stop emitting the radiotherapy rays based on a determination result of whether the first motion amplitude is greater than the threshold.

According to yet another aspect of the present disclosure, a non-transitory computer readable medium may be provided. The non-transitory computer readable medium may comprise at least one set of instructions for MRI-guided radiotherapy. When executed by one or more processors of a computing device, the at least one set of instructions causes the computing device to perform a method. The method may include obtaining first auxiliary signals and second auxiliary signals collected by an MRI device during an MRI scan of a target subject, wherein the first auxiliary signals are collected by the MRI device at a first time before radiotherapy rays are emitted, and the second auxiliary signals are collected by the MRI device at a second time after the radiotherapy rays are emitted. The method may also include determining whether a first motion amplitude of the target subject from the first time to the second time is greater than a threshold based on the first auxiliary signals and the second auxiliary signals. The method may further include determining whether to stop emitting the radiotherapy rays based on a determination result of whether the first motion amplitude is greater than the threshold.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a block diagram illustrating an exemplary processing device according to some embodiments of the present disclosure;

FIG. 3 is a flowchart illustrating an exemplary process for generating real-time MRI images of a subject according to some embodiments of the present disclosure;

FIG. 4 is a schematic diagram illustrating an exemplary MRI pulse sequence for implementing an MRI scan on a subject according to some embodiments of the present disclosure;

FIG. 5 is a flowchart illustrating an exemplary process for determining a temporal factor q; according to some embodiments of the present disclosure;

FIG. 6 is a schematic diagram illustrating an exemplary process for determining a spatial factor U₀ according to some embodiments of the present disclosure; and

FIG. 7 is a schematic diagram illustrating an exemplary process for generating a real-time MRI image of a subject according to some embodiments of the present disclosure;

FIG. 8 is a schematic diagram illustrating an exemplary process for generating a real-time MRI image of a subject according to some embodiments of the present disclosure;

FIG. 9 is a schematic diagram illustrating an exemplary process for generating real-time MRI images of a subject according to some embodiments of the present disclosure;

FIG. 10 is a flowchart illustrating an exemplary process for MRI-guided radiotherapy according to some embodiments of the present disclosure;

FIG. 11 is a schematic diagram illustrating an exemplary MRI pulse sequence for implementing an MRI scan on a target subject according to some embodiments of the present disclosure;

FIG. 12 is a flowchart illustrating an exemplary process for generating a first MRI image during MRI-guided radiotherapy according to some embodiments of the present disclosure;

FIG. 13 is a flowchart illustrating an exemplary process for generating a second MRI image during MRI-guided radiotherapy according to some embodiments of the present disclosure;

FIG. 14 is a flowchart illustrating an exemplary process for generating a third MRI image during MRI-guided radiotherapy according to some embodiments of the present disclosure; and

FIG. 15 is a schematic diagram illustrating an exemplary MRI-guided radiotherapy according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “pixel” and “voxel” in the present disclosure are used interchangeably to refer to an element of an image. Aₙ anatomical structure shown in an image of a subject may correspond to an actual anatomical structure existing in or on the subject's body.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Provided herein are systems and methods for non-invasive biomedical imaging, such as for disease diagnostic or research purposes. While the systems and methods disclosed in the present disclosure are described primarily regarding SMS multitasking imaging using an MRI system. It should be understood that this is only for illustration purposes. The systems and methods of the present disclosure may be applied to any other kind of imaging system. In some embodiments, the imaging system may include a single modality imaging system and/or a multi-modality imaging system. The single modality imaging system may include, for example, the MRI system. The multi-modality imaging system may include, for example, an X-ray imaging-magnetic resonance imaging (X-ray-MRI) system, a single photon emission computed tomography-magnetic resonance imaging (SPECT-MRI) system, a digital subtraction angiography-magnetic resonance imaging (DSA-MRI) system, a computed tomography-magnetic resonance imaging (MRI-CT) system, a positron emission tomography-magnetic resonance imaging (PET-MRI) system, etc.

Conventionally, an MRI image is generated after an MRI scan is finished, and the latency from the start of the MRI scan and the generation of the MRI image is about tens of seconds or even several minutes. Even combined with a compressed sensing technology, a deep learning-based fast imaging technology, or other technologies, conventional MRI technologies still cannot meet the requirements of real-time imaging (e.g., for guiding a radiation therapy).

Recently, MRI techniques based on partially separable low-rank models have been proposed to improve imaging efficiency by acquiring navigation data and imaging data in turn. However, these techniques only can retrospectively construct an MRI image of the subject after the MRI scan is finished, and are not able to construct real-time MRI images in the course of the MRI scan (i.e., when collection of new MRI data continues).

To address the above-mentioned problems of conventional MRI technologies, the present disclosure provides systems and methods for real-time MRI technology. As used herein, the real-time MRI technology refers to a technology that can generate MRI images (i.e., real-time MRI images) with short latency. For example, MRI images may be generated before the MRI scan is finished. In other words, during the MRI scan, real-time MRI images can be generated based on MRI data that has been collected, and at the same time, new MRI data is constantly being collected. As another example, a time period between the collection of MRI signals and the generation of a corresponding MRI image based on the collected MRI signals is shorter than a certain threshold (e.g., 30 million seconds, 40 million seconds, 50 million seconds, 1 seconds, 2 seconds). MRI images acquired using the real-time MRI technology disclosed herein may be referred to as real-time MRI images.

In some embodiments, the systems disclosed herein may determine an initial spatial factor based on first target MRI signals collected in a first imaging stage of an MRI scan of a subject. The systems may also obtain second target MRI signals during the second imaging stage of the MRI scan after the first imaging stage. The systems may determine temporal factors or/and updated spatial factors. Each of the temporal factors may be determined based on the second target MRI signals obtained before the determination of the temporal factor. Each of the updated spatial factors may be determined based on the second target MRI signals obtained before the determination of the updated spatial factor. The systems may further generate real-time MRI images within the second imaging stage. Each of the real-time MRI images may be generated based on the latest determined temporal factor and one of the initial spatial factor or the latest determined spatial factor. A real-time MRI image may reflect a real-time status of the subject in the second imaging stage. More particular, in some embodiments, the first target MRI signals may be collected in the first imaging stage and processed to obtain specific essential data (e.g., an initial temporal factor, a transformation coefficient, and an initial spatial factor). The second imaging stage may include multiple second imaging sub-stages. Each of the multiple second imaging sub-stages may be regarded as a real-time imaging stage for collecting real-time image data (data determined based on second target MRI signals collected in the second imaging sub-stage), which may be used for generating a real-time MRI image corresponding to the second imaging sub-stage. After the second target MRI signals are collected in the second imaging sub-stage, at least a portion of the essential data may be updated, and the real-time MRI image of the subject corresponding to the second imaging sub-stage may be generated based on the updated essential data. Since the second imaging sub-stage is relatively short, an amount of the second target MRI signals collected in the second imaging sub-stage is relatively small, and the generation of the real-time MRI images only involves simple calculation. In this way, real-time imaging can be achieved in the second imaging stage.

MRI is widely used in radiotherapy guidance. In order to meet the requirements of radiotherapy guidance, MRI needs to have a certain degree of real-time performance to capture the change in positions of tumor tissues in time and dynamically adjust a radiotherapy plan to achieve accurate radiotherapy of tissues. In recent years, the technique of spatiotemporal imaging with partially separable functions has been proposed to achieve accelerated imaging. The technique may transform the reconstruction process of dynamic MRI into a process of solving spatial factor and temporal factor, respectively based on the low rank characteristics of dynamic MRI images.

The present disclosure provides a technique of MRI-guided radiotherapy based on spatiotemporal imaging with partially separable functions to improve the real-time performance of MRI during radiotherapy. In some embodiments, the present disclosure uses an additional motion detection device (e.g., an optical camera, a radar device, etc.) to detect a motion of a target subject. In some embodiments, the present disclosure collects auxiliary signals (also referred to as navigation signals) based on spatiotemporal imaging with partially separable functions and detects the motion of the target subject based on the auxiliary signals, which may improve the accuracy and timeliness of motion detection, thereby improving the accuracy of radiotherapy.

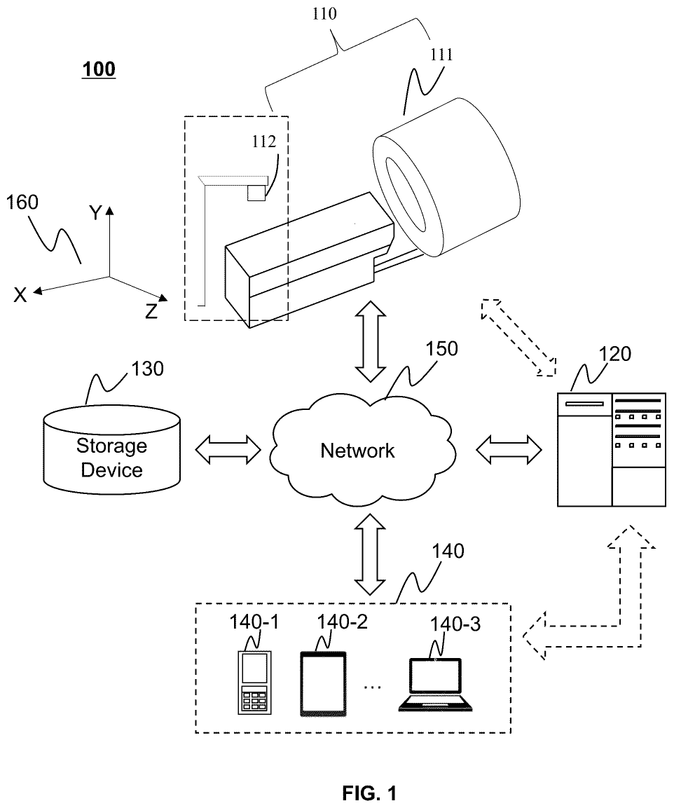

FIG. 1 is a schematic diagram illustrating an exemplary medical system 100 according to some embodiments of the present disclosure. As shown in FIG. 1, the medical system 100 may include a medical device 110, a processing device 120, a storage device 130, one or more terminals 140, and a network 150. In some embodiments, the medical device 110, the processing device 120, the storage device 130, and/or the terminal(s) 140 may be connected to and/or communicate with each other via a wireless connection, a wired connection, or a combination thereof. The connections between the components in the medical system 100 may be variable. For example, the medical device 110 may be connected to the processing device 120 through the network 150. As another example, the medical device 110 may be connected to the processing device 120 directly.

In some embodiments, the medical device 110 may include an MRI scanner 111 and a radiotherapy device 112. In some embodiments, the medical device 110 may include the MRI scanner 111, and the radiotherapy device 112 may be omitted. The MRI scanner 111 may be configured to scan a subject (or a part of the subject) to acquire image data, such as MRI signals (also referred to as MR signals) associated with the subject. For example, the MRI scanner 111 may detect a plurality of MRI signals by applying an MRI pulse sequence on the subject.

In some embodiments, the MRI scanner 111 may be configured to guide the radiotherapy device 112 to perform a radiotherapy treatment on the subject. The MRI scanner 111 may be configured to acquire image data of the subject before radiotherapy treatment, during the radiotherapy treatment, and/or after the radiotherapy treatment. For example, the MRI scanner 111 may be configured to acquire real-time image data of the subject during the radiotherapy treatment. The real-time image data may be used to track motions (e.g., a physiological motion (e.g., cardiac motion), a rigid motion, etc.) of the target and/or one or more organs at risk near the target, so that the delivery of the radiotherapy treatment can be adjusted to adapt to the physiological motions.

In some embodiments, the MRI scanner 111 may include, for example, a main magnet, a gradient coil (or also referred to as a spatial encoding coil), a radio frequency (RF) coil, etc. In some embodiments, the MRI scanner 111 may be a permanent magnet MRI scanner, a superconducting electromagnet MRI scanner, or a resistive electromagnet MRI scanner, etc., according to types of the main magnet. In some embodiments, the MRI scanner 111 may be a high-field MRI scanner, a mid-field MRI scanner, and a low-field MRI scanner, etc., according to the intensity of the magnetic field.

The subject scanned by the MRI scanner 111 may be biological or non-biological. For example, the subject may include a patient, a man-made object, etc. As another example, the subject may include a specific portion, organ, tissue, and/or a physical point of the patient. Merely by way of example, the subject may include head, brain, neck, body, shoulder, arm, thorax, heart, stomach, blood vessel, soft tissue, knee, feet, or the like, or a combination thereof.

For illustration purposes, a coordinate system 160 including an X axis, a Y-axis, and a Z-axis is provided in FIG. 1. The X axis and the Z axis shown in FIG. 1 may be horizontal, and the Y-axis may be vertical. As illustrated, the positive X direction along the X axis may be from the right side to the left side of the MRI scanner 111 seen from the direction facing the front of the MRI scanner 111; the positive Y direction along the Y axis shown in FIG. 1 may be from the lower part to the upper part of the MRI scanner 111; the positive Z direction along the Z axis shown in FIG. 1 may refer to a direction in which the subject is moved out of the scanning channel (or referred to as the bore) of the MRI scanner 111.

The processing device 120 may process data and/or information obtained from the MRI scanner 111, the storage device 130, and/or the terminal(s) 140. For example, the processing device 120 may generate real-time MRI images (also referred to as real-time MR images) of the subject in the process of an MRI scan of the subject based on MRI signals collected by the MRI scanner 111. As another example, after the radiotherapy rays are emitted, the processing device 120 may determine whether stop emitting the radiotherapy ray in real time based on the MRI signals collected by the MRI scanner 111, and generate MRI images in real time. In some embodiments, the processing device 120 may be a single server or a server group. The server group may be centralized or distributed. In some embodiments, the processing device 120 may be local or remote. In some embodiments, the processing device 120 may be implemented by a computing device 300 having one or more components.

The storage device 130 may store data, instructions, and/or any other information. In some embodiments, the storage device 130 may store data obtained from the MRI scanner 111, the processing device 120, and/or the terminal(s) 140. In some embodiments, the storage device 130 may store data and/or instructions that the processing device 120 may execute or use to perform exemplary methods described in the present disclosure. In some embodiments, the storage device 130 may be connected to the network 150 to communicate with one or more other components in the medical system 100 (e.g., the MRI scanner 111, the processing device 120, and/or the terminal(s) 140). One or more components of the medical system 100 may access the data or instructions stored in the storage device 130 via the network 150. In some embodiments, the storage device 130 may be part of the processing device 120 or the terminal(s) 140.

The terminal(s) 140 may be configured to enable user interaction between a user and the medical system 100. For example, during an MR scan of the subject, the terminal(s) 140 may display real-time MRI images of the subject so that a user can know real-time status of the subject. In some occasions, the MR scan of the subject may be performed along with radioactive treatment of the subject, and the user may adjust the delivery of the radioactive treatment based on the real-time MRI images. In some embodiments, the terminal(s) 140 may be connected to and/or communicate with the MRI scanner 111, the processing device 120, and/or the storage device 130. In some embodiments, the terminal(s) 140 may include a mobile device 140-1, a tablet computer 140-2, a laptop computer 140-3, or the like, or a combination thereof. In some embodiments, the terminal(s) 140 may be part of the processing device 120 or the MRI scanner 111.

The network 150 may include any suitable network that can facilitate the exchange of information and/or data for the medical system 100. In some embodiments, one or more components of the medical system 100 (e.g., the MRI scanner 111, the processing device 120, the storage device 130, the terminal(s) 140, etc.) may communicate information and/or data with one or more other components of the medical system 100 via the network 150.

This description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. In some embodiments, the medical system 100 may include one or more additional components and/or one or more components described above may be omitted. Additionally or alternatively, two or more components of the medical system 100 may be integrated into a single component. For example, the processing device 120 may be integrated into the MRI scanner 111. As another example, a component of the medical system 100 may be replaced by another component that can implement the functions of the component. However, those variations and modifications do not depart the scope of the present disclosure.

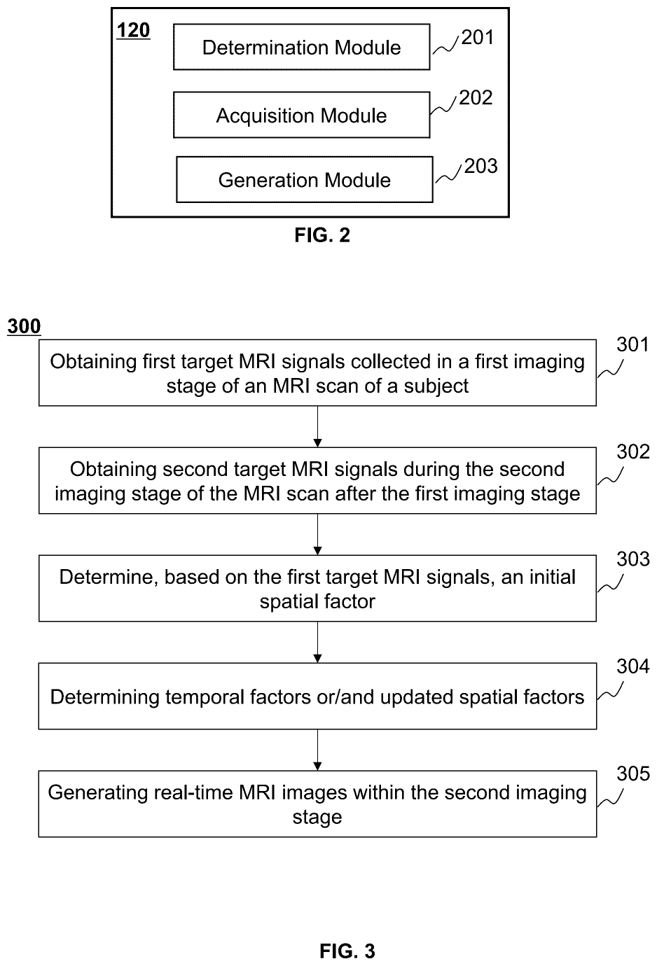

FIG. 2 is a block diagram illustrating an exemplary processing device 120 according to some embodiments of the present disclosure. In some embodiments, the processing device 120 may be implemented on a processing unit (e.g., a processor of a computing device or a CPU of a terminal). As shown in FIG. 2, the processing device 120 may include a determination module 201, an acquisition module 202, and a generation module 203.

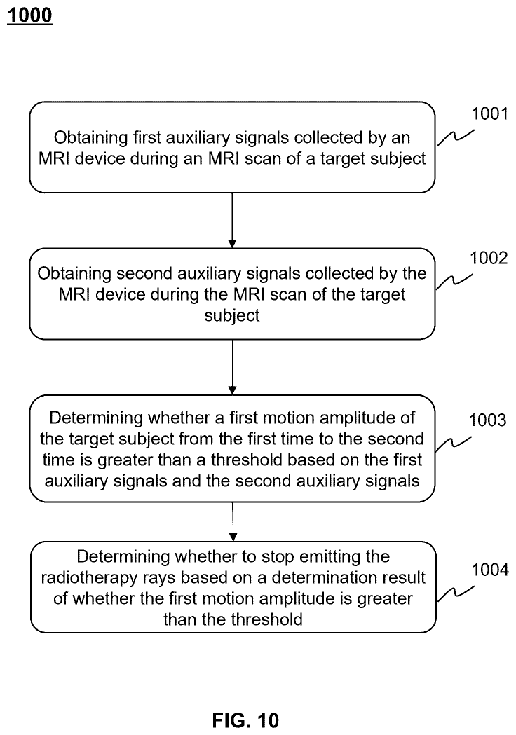

The acquisition module 202 may be configured to obtain information relating to the medical system 100. For example, the acquisition module 202 may obtain first target MRI signals collected in a first imaging stage of an MRI scan of a subject. As another example, the acquisition module 202 may obtain second target MRI signals during the second imaging stage of the MRI scan after the first imaging stage. More descriptions regarding the obtaining of the first target MRI signals and the second target MRI signals may be found elsewhere in the present disclosure. See, e.g., operations 301 and 302 in FIG. 3, and relevant descriptions thereof. As still another example, the acquisition module 202 may obtain first auxiliary signals collected by an MRI device during an MRI scan of a target subject, and second auxiliary signals collected by the MRI device during the MRI scan of the target subject. More descriptions regarding the obtaining of the first auxiliary signals and the second auxiliary signals may be found elsewhere in the present disclosure. See, e.g., operations 1001 and 1002 in FIG. 10, and relevant descriptions thereof.

The determination module 201 may be configured to determine an initial spatial factor based on the first target MRI signals. In some embodiments, the determination module 201 may determine the initial temporal factor based on first target auxiliary signals of the first target MRI signals. More descriptions regarding the determination of the initial spatial factor based on the first target MRI signals may be found elsewhere in the present disclosure. See, e.g., operation 303 in FIG. 3, and relevant descriptions thereof.

In some embodiments, the determination module 201 may be configured to determine temporal factors or/and updated spatial factors. In some embodiments, each of the temporal factors may be determined based on the second target MRI signals collected before the determination of the temporal factor. In some embodiments, each of the updated spatial factors may be determined based on the second target MRI signals obtained before the determination of the updated spatial factor. More descriptions regarding the determination of the temporal factors or/and updated spatial factors may be found elsewhere in the present disclosure. See, e.g., operation 304 in FIG. 3, and relevant descriptions thereof. In some embodiments, the determination module 201 may be configured to determine whether a first motion amplitude of the target subject from the first time to the second time is greater than a threshold based on the first auxiliary signals and the second auxiliary signals. More descriptions regarding the determination of whether the first motion amplitude of the target subject from the first time to the second time is greater than the threshold may be found elsewhere in the present disclosure. See, e.g., operation 1003 in FIG. 10, and relevant descriptions thereof. In some embodiments, the determination module 201 may be configured to determine whether to stop emitting the radiotherapy rays based on a determination result of whether the first motion amplitude is greater than the threshold. More descriptions regarding the determination of whether to stop emitting the radiotherapy rays may be found elsewhere in the present disclosure. See, e.g., operation 1004 in FIG. 10, and relevant descriptions thereof.

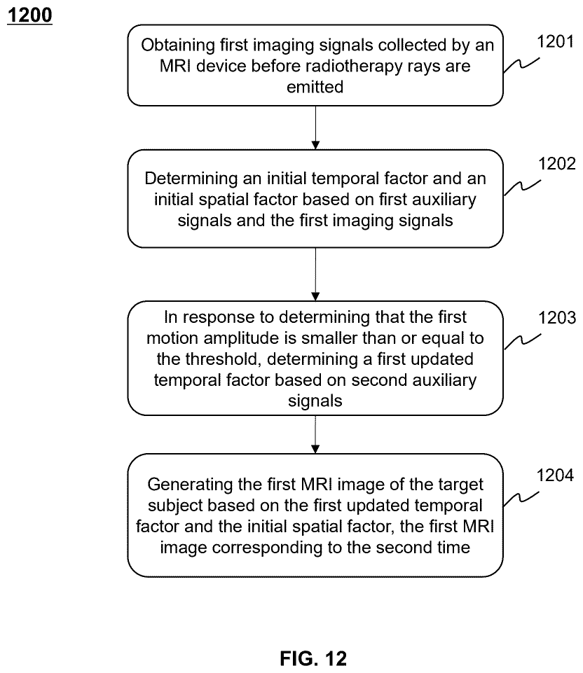

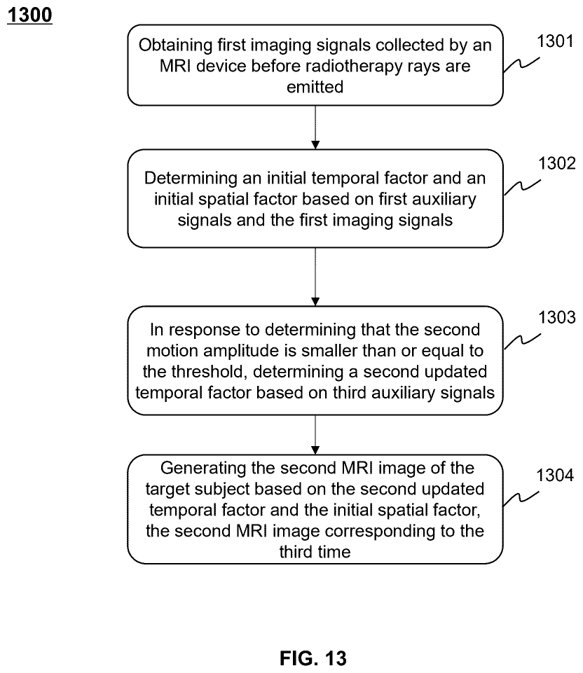

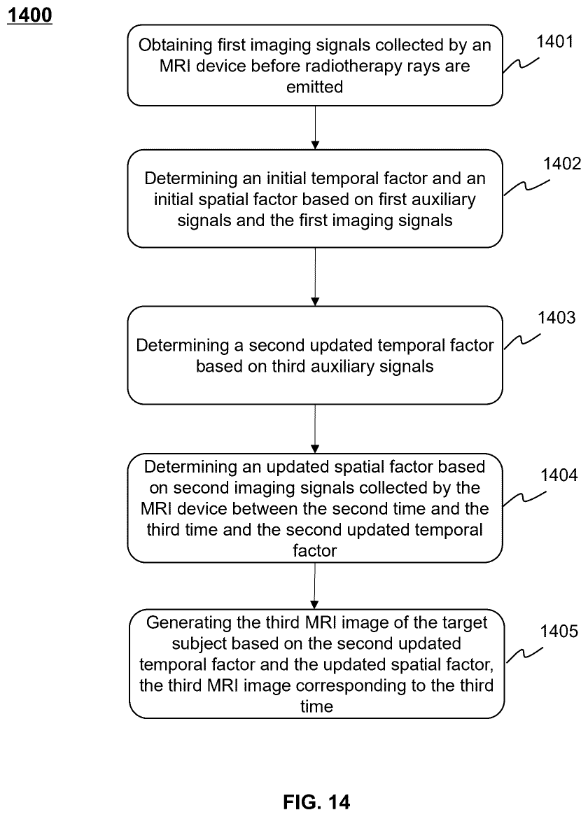

The generation module 203 may be configured to generate real-time MRI images within the second imaging stage. A real-time MRI image may reflect a real-time status of the subject in a corresponding second imaging sub-stage. More descriptions regarding the generation of the real-time MRI images may be found elsewhere in the present disclosure. See, e.g., operation 305 in FIG. 3, and relevant descriptions thereof. In some embodiments, the generation module 203 may be configured to generate a first MRI image, a second MRI image, or a third MRI image of the target subject. More descriptions regarding the generation of the first MRI image, the second MRI image, and the third MRI image of the target subject may be found elsewhere in the present disclosure. See, e.g., operation 1204 in FIG. 12, operation 1304 in FIG. 13, operation 1405 in FIG. 14, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the processing device 120 may further include a storage module (not shown in FIG. 2). The storage module may be configured to store data generated during any process performed by any component of the processing device 120. As another example, each of at least some components of the processing device 120 may include a storage apparatus. Additionally or alternatively, at least some components of the processing device 120 may share a common storage apparatus.

FIG. 3 is a flowchart illustrating an exemplary process 300 for generating real-time MRI images of a subject according to some embodiments of the present disclosure. In some embodiments, the process 300 may be implemented in the medical system 100 illustrated in FIG. 1. For example, the process 300 may be stored in a storage device of the MRI system as a form of instructions, and invoked and/or executed by the processing device 120 (e.g., one or more modules as illustrated in FIG. 2). The operations of the illustrated process presented below are intended to be illustrative. In some embodiments, the process 300 may be accomplished with one or more additional operations not described, and/or without one or more of the operations discussed. Additionally, the order in which the operations of the process 300 as illustrated in FIG. 3 and described below is not intended to be limiting.

As used herein, the subject may be biological or non-biological. For example, the subject may include a patient, a man-made object, etc. As another example, the subject may include a specific portion, organ, tissue, and/or a physical point of the patient. Merely by way of example, the subject may include head, brain, neck, body, shoulder, arm, thorax, heart, stomach, blood vessel, soft tissue, knee, feet, or the like, or a combination thereof.

As aforementioned, a real-time MRI image refers to an MRI image obtained using a real-time imaging technology. For example, the real-time MRI image may be generated when the MRI scan is still performed. As another example, the latency between the generation of the real-time MRI image and the collection of corresponding MRI signals may be shorter than a certain threshold.

In 301, the processing device 120 (e.g., the acquisition module 202) may obtain first target MRI signals collected in a first imaging stage of an MRI scan of a subject.

In 302, the processing device 120 (e.g., the acquisition module 202) may obtain second target MRI signals during the second imaging stage of the MRI scan after the first imaging stage.

In some embodiments, the MRI scan of the subject may include the first imaging stage (or referred to as the first imaging phase) and the second imaging stage (or referred to as a second imaging phase) after the first imaging stage. The first imaging stage may be regarded as a training stage or a preparation stage for collecting essential data (data determined based on the first target MRI signals, such as an initial temporal factor (denoted as φ₀), a transformation coefficient, and an initial spatial factor (denoted as U₀, which may serve as a basis for achieving real-time imaging in the second imaging stage.

In some embodiments, the second imaging stage may include multiple second imaging sub-stages, and operation 302 may be performed for each second imaging sub-stage. Each of the multiple second imaging sub-stages may be regarded as a real-time imaging stage for collecting real-time image data (data determined based on second target MRI signals collected in the second imaging sub-stage), which may be used for generating a real-time MRI image corresponding to the second imaging sub-stage. For example, the processing device 120 may obtain second target MRI signals {M₁, M₂, . . . , Mₙ}. The second target MRI signals Mᵢ may be collected at time Tᵢ among time series {T₁, T₂, . . . , In} during the second imaging stage. Tᵢ may be a relatively small period of time and correspond one second imaging sub-stage described in operation 302, that is, the second target MRI signals Mᵢ may be collected at one second imaging sub-stage. i may be an integer greater than 0 and smaller than n, and n may be an integer greater than 1.

In some embodiments, the essential data may be determined before the second imaging stage. For example, there may be a time interval between the first and second imaging stages for determining the essential data based on the first target MRI signals. Alternatively, the second imaging stage may begin immediately after the first imaging stage, that is, there is no time interval between the first and second imaging stages. In such cases, the determination of the essential data may be performed during the second imaging stage (i.e., along with the acquisition of the second target MRI signals).

In some embodiments, the durations of the first imaging stage and the second imaging sub-stages may be set manually by a user (e.g., an engineer) according to an experience value or a default setting of the medical system 100. Additionally or alternatively, the durations of the first imaging stage and the imaging stage may be determined by the processing device 120 according to an actual need (e.g., requirements on the total scan time, the imaging quality, etc.) Merely by way of example, the first imaging stage may last for 30 seconds, 40 seconds, 60 seconds, or the like, and the second imaging sub-stage may last for such as 50 milliseconds, 100 milliseconds, 150 milliseconds, or the like. In some embodiments, the second imaging sub-stage may be much smaller than the first imaging stage. For example, the first imaging stage may be greater than 60 seconds and the second imaging sub-stage may be smaller than 1 seconds. In such cases, sufficient and accurate essential data can be obtained in the first imaging stage, and the real-time imaging may be achieved in the second imaging stage.

In some embodiments, in the MRI scan, an MRI scanner (e.g., the MRI scanner 111) may apply an MRI pulse sequence to the subject and collect MRI signals from the subject. The MRI signals collected in the first imaging stage may be referred to as the first target MRI signals, and the MRI signals collected in the second imaging stage may be referred to as the second target MRI signals. The MRI pulse sequence may be of any type of MRI pulse sequences, such as a spin echo sequence, a gradient echo sequence, a diffusion sequence, an inversion recovery sequence, or the like, or any combination thereof.

In some embodiments, the processing device 120 may obtain the first target MRI signals and the second target MRI signals from an MRI scanner for performed the MRI scan of the subject or a storage device that stores the first target MRI signals.

In some embodiments, the first target MRI signals may include a plurality of first target auxiliary signals and a plurality of first target imaging signals, and the second target MRI signals may include a plurality of second target auxiliary signals. In some embodiments, the second target MRI signals may further include a plurality of second target imaging signals.

An auxiliary signal may also be referred to as a navigator signal, and include high-temporal resolution data relating to at least one time-varying dimension of the subject. The at least one time-varying dimension may include any dimension that reflects time-varying characteristics or dynamic information of the subject. In some embodiments, the at least one time-varying dimension of the subject may include a dimension relating to an elapsed time. In some embodiments, the at least one time-varying dimension of the subject may include one or more dimensions relating to other information, such as a cardiac motion, a respiratory motion, a T1 relaxation, a T2 relaxation, a chemical exchange saturation transfer (CEST), a contrast agent dynamic, a T1ρ contrast, a molecular diffusion, etc.

In some embodiments, the first target auxiliary signals and the second target auxiliary signals may correspond to the same subset of K-space (e.g., which includes one or more K-space lines) and collected by sampling the subset of K-space repeatedly with a high sampling frequency. For example, the first target auxiliary signals and the second target auxiliary signals may correspond to the same K-space line in K-space and be acquired by sampling the K-space line repeatedly with a high sampling frequency. As used herein, a high sampling frequency refers to a sampling frequency that is higher than a threshold frequency. The threshold frequency may be a default value, or determined manual by a user, or determined by the processing device 120 according to data analysis. For example, the threshold frequency may be determined according to the at least one time-varying dimension to be analyzed. Merely by way of example, a time-varying dimension may relate to the respiratory motion of the subject, and the respiration cycle of the subject is close to 0.75 seconds(s). In order to capture dynamic information relating to the respiratory motion of the subject, the sampling frequency may need to be greater than a threshold frequency of 1/0.75 Hertz (HZ). As another example, the threshold frequency may be determined according to actual requirements, experience, a data model, etc.

An imaging signals may include high-spatial resolution image data relating to at least one spatial-varying dimension of the subject. Exemplary spatial-varying dimensions may relate to a phase encoding direction, a frequency encoding direction, or the like, or any combination thereof. In some embodiments, the first target imaging signals and the second target MRI signals may be acquired using a pseudo-random trajectory collection manner by sampling different K-space lines in K-space.

The first target auxiliary signals, the first target imaging signals, the second target auxiliary signals, and the second target imaging signals may be acquired by any suitable sampling pattern. For illustration purposes, the acquisition of the first target auxiliary signals and the first target imaging signals are described hereinafter. The second target auxiliary signals may be acquired in a similar manner to the first target auxiliary signals, and the second target imaging signals may be acquired in a similar manner to the first target imaging signals. In some embodiments, the first target auxiliary signals and the first target imaging signals may be acquired by radial sampling. The first target auxiliary signals may correspond to a radial line in K-space of a constant angle (e.g., 0°, 10°, 20°, 30°, 100°, 180°, etc.). Merely by way of example, the first target auxiliary signals may be acquired by sampling a radial line in the K-space of the constant angle repeatedly at a regular interval. The first target imaging signals may correspond to a plurality of radial lines in K-space of different readout angles. In some embodiments, the first target imaging signals may be acquired by sampling the plurality of radial lines in K-space according to a golden-angle radial sampling schedule. By adopting the golden-angle radial sampling schedule, multiple radial spokes that are uniformly distributed in and cover K-space can be acquired in a relatively short time, which may improve the scanning efficiency and reduce the computation amount and the computation time. It should be understood that the first target imaging signals may be sampled by any readout angle (e.g., randomly set readout angles) according to an actual need (e.g., based on the requirement(s) regarding the scanning time and/or the imaging quality).

In some embodiments, the first target auxiliary signals and the first target imaging signals may be acquired by Cartesian sampling. The first target auxiliary signals may correspond to the same Cartesian line in K-space, and the first target imaging signals may correspond to different Cartesian lines in K-space. In some embodiments, the first target auxiliary signals may correspond to the Cartesian line passing through a K-space center in K-space. In some embodiments, the first target imaging signals may be acquired by Cartesian sampling while the first target auxiliary signals may be acquired by sampling a specific radial line or spiral line in K-space repeatedly.

The first target auxiliary signals and the first target imaging signals may be acquired in any sampling order during the MRI scan of the subject. In some embodiments, the first target auxiliary signals and the first target imaging signals may be acquired interleaved during the MRI scan of the subject. For example, a first count of first target imaging signals may be sampled after or before every readout of a second count of first target auxiliary signals. The first count and the second count may be any positive integer, such as 1, 2, 3, 5, 10, etc. In some embodiments, the first count and the second count may be set according to actual requirements, for example, a sampling frequency of the first target auxiliary signals needs to be greater than the threshold frequency and/or enough first target imaging signals need to be acquired for image reconstruction. In some embodiments, the ratio of the first count to the second count may relate to the type of the subject to be imaged. For example, to image the heart of a patient, the ratio of the first count to the second count may be equal to 1:1. As another example, to image an organ other than the heart (e.g., an arm, a knee), the ratio of the first count to the second count may be equal to 10:1. In some embodiments, the first target auxiliary signals may have no phase encoding, which may be used to estimate temporal factors relating to the at least one time-varying dimension. Phase encodings of the first target imaging signals may conform to a certain rule (e.g., a random Gaussian distribution).

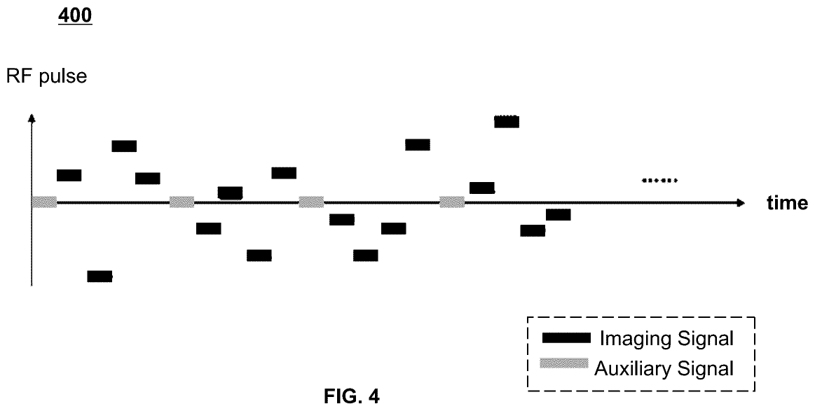

Merely by way of example, FIG. 4 is a schematic diagram illustrating an exemplary MRI pulse sequence 400 for implementing an MRI scan on a subject according to some embodiments of the present disclosure. As shown in FIG. 4, four first target imaging signals are acquired after every readout of one first target auxiliary signal. In such cases, a ratio of the count of the first target imaging signals to the count of the first target auxiliary signals obtained in operation 301 may be 4:1. The first target auxiliary signals have no phase encoding and are repeated periodically. Phase encodings of the first target imaging signals conform to a random Gaussian distribution.

In some embodiments, the auxiliary signals and the imaging signals may have a preset readout direction, respectively. The readout direction may be a default setting of the system, or may be set by a user, or may be determined by the processing device 120 according to actual conditions. For example, when the target region receiving radiotherapy has a physiological motion, the readout direction of the auxiliary signals may be related to a motion direction of the target region. The motion direction of the target region refers to a direction in which a motion amplitude (or a motion displacement) of the target region has a detectable change during the physiological motion. In some embodiments, the readout direction of the auxiliary signals may be parallel or substantially parallel to the motion direction of the target region. For example, for a respiratory motion, the motion direction may be a head-to-foot direction, and the readout direction of the auxiliary signals may be parallel or substantially parallel to the head-to-foot direction of the target region.

In some embodiments, the readout direction of the imaging signals may be a direction with relatively high imaging efficiency. In some embodiments, it can be considered that in a single scan, the larger an area of a scan region to be scanned covered by a field of view (FOV) of the MRI device, the higher the imaging efficiency. For example, if the MRI scan (e.g., a liver scan) is performed to acquire images of axial planes of the target subject, the readout direction of the imaging data may be a left-right direction of the target subject. As another example, if the MRI scan is performed to acquire images of coronal planes of the target subject, the readout direction of the imaging data may be an up-down direction of the target subject.

In 303, the processing device 120 (e.g., the determination module 201) may determine, based on the first target MRI signals, an initial spatial factor (denoted as U₀).

In some embodiments, operation 303 may be performed before operation 302 or at the same time with operation 302.

In some embodiments, the processing device 120 may determine an initial temporal factor φ₀ based on the first target auxiliary signals. In some embodiments, a temporal factor may include one or more temporal basis functions relating to the elapsed time. Each temporal basis function may relate to a time-varying dimension of the subject. In some embodiments, the temporal factor may include one or more cardiac temporal basis functions relating to the cardiac motion of the subject, one or more respiratory temporal basis functions relating to the respiratory motion of the subject, one or more T₁ recovery temporal basis functions relating to the T₁ relaxation of the subject, or the like, or any combination thereof. A temporal basis function relating to a time-varying dimension may reflect dynamic information along the time-varying dimension and include high-temporal resolution information.

In some embodiments, the processing device 120 may determine a transformation coefficient T and the initial temporal factor φ₀ based on the first target auxiliary signals. The transformation coefficient T may represent a relationship between auxiliary signals and temporal factors. In some embodiments, the plurality of first target auxiliary signals may be filled into K-space to obtain a first K-space matrix. The processing device 120 may determine the transformation coefficient T and the initial temporal factor φ₀ based on the first K-space matrix. For example, the processing device 120 may determine the transformation coefficient T and the initial temporal factor do according to a singular value decomposition (SVD) algorithm. Merely by way of example, the first K-space matrix may be denoted as K₁. The first K-space matrix K₁ may be presented as

$$( \begin{matrix}κ ( {k}_{1} , {t}_{1} ) & … & κ ( {k}_{1} , {t}_{N} ) \\ κ ( {k}_{2} , {t}_{1} ) & … & κ ( {k}_{2} , {t}_{N} ) \\ ⋮ & … & ⋮ \\ κ ( {k}_{c} , {t}_{1} ) & … & κ ( {k}_{c} , {t}_{N} )\end{matrix} ) ,$$

wherein an element in the first K-space matrix K₁ represents k-space data collected by a specific coil channel at a certain moment. For example, κ(k_{c}, t₁) represents k-space data collected by the coil channel k_{c} at a moment t₁. The processing device 120 may determine the transformation coefficient T and the initial temporal factor do by performing the SVD on the first K-space matrix K₁ according to Equation (1) as below:

$${K}_{1} = {U}_{k} D {φ}_{0} , \qquad ( 1 )$$

where Uₖ denotes a projection coefficient matrix, φ₀ denotes the initial temporal factor (e.g., in the form of at least one temporal factor matrix), D denotes a singular value matrix. The transformation coefficient T may be determined based on the projection coefficient matrix Uₖ and the singular value matrix D. For example, the transformation coefficient T may be (UₖD) or

$$( {D}^{- 1} {U}_{k}^{H} )$$

(in which

$${U}_{k}^{H}$$

denotes a conjugate transpose matrix of the projection coefficient matrix Uₖ, and D⁻¹ denotes an inverse matrix of the singular value matrix D).

Further, the processing device 120 may determine the initial spatial factor U₀ based on the initial temporal factor φ₀ and the first target imaging signals. A spatial factor of the subject may include high-spatial resolution information along a spatial-varying dimension. For example, the spatial factor may reflect a relationship between pixel information of the subject in the image domain and spatial information of the subject in the physical domain. In some embodiments, the spatial factor may be represented as a basis image that includes high-spatial resolution information. Different spatial factors may be represented as basis images that include different high-spatial resolution information.

In some embodiments, the processing device 120 may construct an optimization function relating to the initial spatial factor U₀. The optimization function may incorporate the plurality of first target imaging signals and the initial temporal factor φ₀. The processing device 120 may further determine the initial spatial factor U₀ by solving the optimization function. For example, the processing device 120 may determine the initial spatial factor U₀ of the subject according to a first optimization function shown in Equation (2) as below:

$$= {arg min}_{{U}_{0}} {\sum }_{j = 1}^{c} {‖ Ω {FS}_{j} {U}_{0} {Φ}_{0} - {d}_{j} ‖}_{2}^{2} , \qquad ( 2 )$$

where [symbol]
denotes the optimal spatial factor determined by solving Equation (2), U₀ denotes the initial spatial factor of the subject (e.g., in the form of at least one spatial factor matrix), c denotes the count of coil channels of the MRI scanner, dⱼ denotes K-space data obtained by filling first target imaging signals acquired by the jth coil channel into the K-space, Ω denotes an undersampling operator (which may be omitted in some conditions), F denotes a Fourier transformation operator, Sⱼ denotes a coil sensitive map corresponding to the j^{th} coil channel, Φ₀ denotes the initial temporal factor φ₀ of the subject (e.g., in the form of at least one temporal factor matrix).

As another example, the processing device 120 may determine the initial spatial factor U₀ of the subject according to a second optimization function shown in Equation (3) as below:

$$= {arg min}_{{U}_{0}} {\sum }_{j = 1}^{c} {‖ Ω {FS}_{j} {U}_{0} {Φ}_{0} - {d}_{j} ‖}_{2}^{2} + λ {‖ TV ( {U}_{0} ) ‖}_{1} , \qquad ( 3 )$$

where λ denotes a regularization parameter, TV(U₀) denotes a 3-dimensional total variation, and λ∥TV(U₀)∥₁ denotes a constraint item relating to the at least one spatial factor matrix of the subject (which may be omitted in some conditions).

In 304, the processing device 120 (e.g., the determination module 201) may determine temporal factors or/and updated spatial factors.

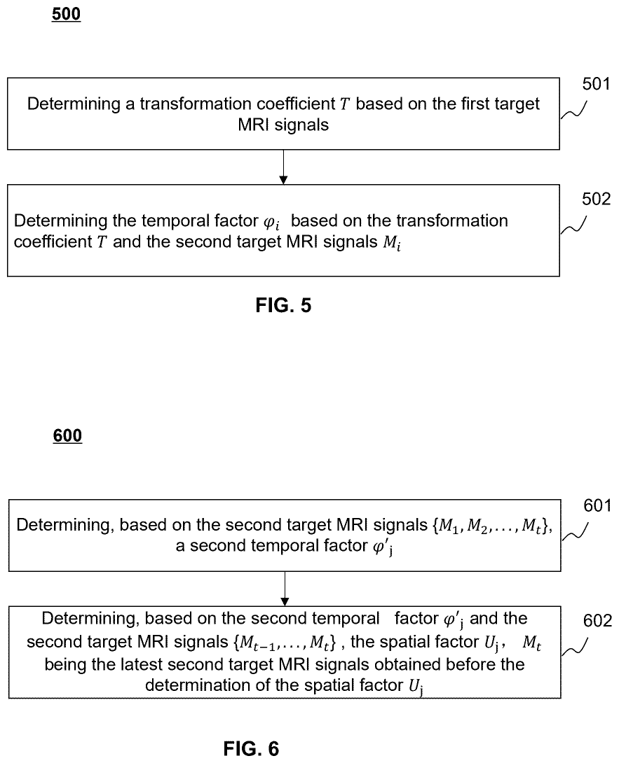

In some embodiments, each of the temporal factors may be determined based on the second target MRI signals collected before the determination of the temporal factor. Merely by way of example, the second target MRI signals may include the second target MRI signals {M₁, M₂, . . . , Mₙ} described in the operation 302, and the processing device 120 may determine temporal factors {φ₁, φ₂, . . . , φₙ} (or referred to as first temporal factors) based on the second target MRI signals {M₁, M₂, . . . , Mₙ}. The temporal factor φᵢ may be determined based on the second target MRI signals Mᵢ, wherein i may be an integer greater than 0, and smaller than or equal to n. In some embodiments, the temporal factor φᵢ may be determined based on the second target MRI signals Mᵢ within a first time period. The first time period may be, for example, 40 milliseconds, 50 milliseconds, 100 milliseconds, 150 milliseconds, etc. In such cases, a second imaging sub-stage as aforementioned may last for the first time period (e.g., 50 milliseconds). More descriptions for the determination of the temporal factor φᵢ may be found elsewhere in the present disclosure (e.g., FIG. 5 and the descriptions thereof).

In some embodiments, each of the updated spatial factors may be determined based on the second target MRI signals obtained before the determination of the updated spatial factor. Alternatively, an updated spatial factor may be determined based on at least a portion of the first target MRI signals and the second target MRI signals obtained before the determination of the updated spatial factor. Merely by way of example, the processing device 120 may determine the spatial factors {U₁, U₂, . . . , Uₘ} based on the second target MRI signals {M₁, M₂, . . . , Mₙ}, wherein m may be smaller than or equal to n. The spatial factor U; may be determined based on the second target MRI signals {M₁, M₂, . . . , Mₜ}, wherein 1<t<m, j may be an integer greater than 0, and smaller than or equal to m, and Mₜ is the latest second target MRI signals obtained before the determination of Uⱼ. In some embodiments, the spatial factor U; may be determined based on the second target MRI signals {M₁, M₂, . . . , Mₜ} within a second time period. The second time period may be, for example, 400 milliseconds, 500 milliseconds, 1 second, etc. More descriptions for the determination of the spatial factor may be found elsewhere in the present disclosure (e.g., FIG. 6 and the descriptions thereof).

In some embodiments, the processing device 120 may monitor body motion of the subject during the second imaging stage. In some embodiments, the processing device 120 may obtain motion information of the subject. In some embodiments, the motion information of the subject may include a magnitude of the motion of the object from the first imaging stage to one second imaging sub-stage in the second imaging stage. The magnitude of the motion of the object from the first imaging stage to the second imaging stage may be represented by a magnitude of the motion of the object from a first moment of the first imaging stage to a second moment of the second imaging sub-stage. The first moment may be any moment of the first imaging stage, and the second moment may be any moment of the second imaging sub-stage. For example, the first moment may be a starting moment of the first imaging stage, and the second moment may be an ending moment of the second imaging sub-stage. As another example, the first moment may be an ending moment of the first imaging stage, and the second moment may be an ending moment of the second imaging sub-stage. In some embodiments, an interval between the first moment and the second moment may be greater than a time threshold. The first moment, the second moment, and the time threshold may be set manually by a user (e.g., an engineer) according to an actual need or a default setting of the medical system 100.

In some embodiments, the motion information of the subject may be determined based on a first image and a second image of the subject captured by an image acquisition device at the first moment and the second moment, respectively. The image acquisition device may be and/or include any suitable device that is capable of capturing image data of subjects located in a field of view of the image acquisition device. For example, the image acquisition device may include a camera (e.g., a digital camera, an analog camera, a depth camera, a structured light camera, etc.), a red-green-blue (RGB) sensor, an RGB-depth (RGB-D) sensor, a Lidar, or the like, or any combination thereof. For example, the processing device 120 may the first image and the second image. The processing device 120 may determine the motion information of the subject by analyzing positions of the subject in the first image and the second image.

In some embodiments, the motion information of the scanned subject may be determined based on the auxiliary signals. More descriptions regarding the determination of the motion information based on the auxiliary signals may be found in FIG. 10, which are not repeated here.

In some embodiments, the motion information of the subject may be determined by other manners. For example, the motion information of the subject may be determined based on distance information from the body surface of the subject to a reference location, wherein the distance information may be collected using a microwave technology or an ultrasonic technology. In some embodiments, the processing device 120 may determine a maximum magnitude of the motion of the subject from the first imaging stage to the second imaging stage, and designate the maximum magnitude of the motion of the subject as the final magnitude of the motion of the subject from the first imaging stage to the second imaging sub-stage.

In response to detecting that a magnitude of the monitored body motion of the subject exceeds a magnitude threshold, which indicates that the subject has an obvious motion from the first imaging stage to the second imaging sub-stage, the processing device 120 may determine a next spatial factor among the spatial factors {U₁, U₂, . . . , Uₘ}, that is, a current spatial factor needs to be updated. In some embodiments, in response to detecting that the magnitude of the monitored body motion of the subject does not exceed the magnitude threshold, which indicates that the subject does not have an obvious motion from the first imaging stage to the second imaging sub-stage, that is, the current spatial factor does not need to be updated.

In 305, the processing device 120 (e.g., the generation module 202) may generate real-time MRI images within the second imaging stage.

As aforementioned, the second imaging stage may include multiple second imaging sub-stages. The real-time MRI images may include a plurality of real-time MRI images each of which corresponds to one second imaging sub-stage. A real-time MRI image may reflect a real-time status of the subject in a corresponding second imaging sub-stage. For example, the real-time MRI image may be generated within a short time (e.g., shorter than a preset period) with little latency after second target MRI signals are collected in the second imaging sub-stage. As another example, the real-time MRI images may be generated before the MRI scan is finished (e.g., MRI signal acquisition is still performed). In some embodiments, the real-time MRI image of the subject may be a two-dimension (2D) image, a three-dimension (3D) image, etc.

In some embodiments, each of the real-time MRI images may be generated based on the latest determined temporal factor and one of the initial spatial factor or the latest determined spatial factor. In some embodiments, each time a temporal factor is determined, a real-time MRI image may be generated based on the determined temporal factor and one of the initial spatial factor or the latest determined spatial factor. Each of the real-time MRI images may reflect the status of the subject at a corresponding second imaging sub-stage. Merely by way of example, the processing device 120 may generate real-time MRI images {A₁, A₂, . . . , Aₙ}. The real-time MRI image Aᵢ may reflect the status of the subject at the second imaging sub-stage corresponding to the time Tᵢ. The real-time MRI image Aᵢ may be generated based on the temporal factor φᵢ and one of the initial spatial factor U₀ or the latest spatial factor determined before the time Tᵢ. In some embodiments, if the initial spatial factor has not been updated before the time Tᵢ (or the generation of the real-time MRI image Aᵢ), the initial spatial factor may be used to generate the real-time MRI image Aᵢ. If the initial spatial factor has been updated before the time Tᵢ (or the generation of the real-time MRI image Aᵢ), the lasted updated spatial factor may be sued to generate the real-time MRI image Aᵢ.

For illustration purposes, exemplary method for generating the real-time MRI image Aᵢ are provided hereinafter. In some embodiments, the real-time MRI image Aᵢ of the subject may be represented by a multi-dimensional tensor, which may be determined based on the temporal factor φᵢ and the initial spatial factor U₀. For example, with the temporal factor φᵢ and the initial spatial factor U₀ available, the processing device 120 may generate the real-time MRI image Aᵢ of the subject by determining a product of at least one temporal factor matrix including the temporal factor φᵢ and at least one spatial factor matrix including the initial spatial factor U₀. Merely by way of example, the processing device 120 may generate the real-time MRI image Aᵢ of the subject according to Equation (4) as below:

$${A}_{i} = {U}_{0} {φ}_{i} , \qquad ( 4 )$$

where Aᵢ denotes a multi-dimensional tensor for representing the real-time MRI image Aᵢ of the subject, φᵢ denotes the temporal factor in the form of the at least one temporal factor matrix, and U₀ denotes the initial spatial factor in the form of the at least one spatial factor matrix.

In some embodiments, the processing device 120 may generate the real-time MRI image Aᵢ of the subject corresponding to a certain time-varying dimension based on the temporal factor matrix corresponding to the certain time-varying dimension and the at least one spatial factor matrix including the spatial factor φᵢ. For example, the processing device 120 may generate a real-time MRI image of the heart by determining a product of at least one spatial factor matrix including the spatial factor of the heart and the temporal factor matrix including the temporal factor relating to the cardiac motion.

In some embodiments, the processing device 120 may generate the real-time MRI image Aᵢ of the subject based on the temporal factor φᵢ, the initial spatial factor U₀, and a core tensor. The core tensor may govern the interaction between the at least one temporal factor matrix and the at least one spatial factor matrix. For example, the processing device 120 may generate the real-time MRI image Aᵢ of the subject by determining a product of at least one temporal factor matrix including the temporal factor φᵢ, at least one spatial factor matrix including the initial spatial factor U₀, and the core tensor. In some embodiments, the core tensor may be determined based on the first target auxiliary signals.

In some embodiments, during the real-imaging of the subject, the processing device 120 may only need to determine the value of the temporal factor φᵢ, and the real-time MRI image Aᵢ may be generated based on the temporal factor φᵢ and the initial determined spatial factor U₀. The generation of the real-time MRI image Aᵢ may involve simple calculation and thereby having a short latency. In some occasions, the determination of the essential data may be performed immediately after the first target MRI signals are collected (e.g., before the second imaging stage or along with the collection of the second target MRI signals), and in this way, the image generation latency in the second imaging stage may be further reduced.

In some embodiments, the processing device 120 may generate the real-time MRI image Aᵢ of the subject based on the temporal factor φᵢ and the latest spatial factor determined before the time Tᵢ in a similar manner as how the real-time MRI image Aᵢ of the subject is generated based on the temporal factor φᵢ and the initial spatial factor U₀. Merely by way of example, the processing device 120 may generate the real-time MRI image Aᵢ of the subject according to Equation (5) as below:

$${A}_{i} = {U}_{j} {φ}_{i} \qquad ( 5 )$$

where Aᵢ denotes a multi-dimensional tensor for representing the real-time MRI image Aᵢ of the subject, φᵢ denotes the temporal factor in the form of at least one temporal factor matrix, and U; denotes the latest spatial factor determined before the time Tᵢ in the form of the at least one spatial factor matrix.

As aforementioned, during the real-imaging of the subject, except the temporal factor φᵢ, the processing device 120 may also determine the value of the latest spatial factor Uⱼ. The processing device 120 may further generate the real-time MRI image Aᵢ of the subject based on the temporal factor φᵢ and the latest spatial factor Uⱼ. In some embodiments, by monitoring the body motion of the subject and updating the current spatial factor when the subject has an obvious motion, the imaging quality of the generated real-time MRI image Aᵢ may be improved (e.g., by reducing motion artifacts).

In some embodiments, real-time MRI images corresponding to multiple second imaging sub-stages may be generated to form a dynamic image. The dynamic image may reflect dynamic information of the subject along a time-varying dimension. For example, a dynamic image may reflect the cardiac motion of the heart over a cardiac cycle, and include a plurality of real-time MRI images of the heart corresponding to a plurality of cardiac phases in the cardiac cycle.

Conventionally, an MRI image is generated after an MRI scan is finished, and the latency from the start of the MRI scan and the generation of the MRI image is about tens of seconds or even several minutes. According to some embodiments of the present disclosure, the first target MRI signals may be collected in the first imaging stage and processed to obtain specific essential data (e.g., the initial temporal factor, the transformation coefficient, and the initial spatial factor). After the second target MRI signals are collected in a second imaging sub-stage, at least a portion of the essential data may be updated, and the real-time MRI image of the subject corresponding to the second imaging sub-stage may be generated based on the updated essential data. Since the second imaging sub-stage is relatively short, an amount of the second target MRI signals is relatively small, and the generation of the real-time MRI image only involves simple calculation (e.g., matrix multiplication and/or matrix division). In this way, real-time MRI imaging can be achieved in the second imaging stage.

In some embodiments, the process 300 may be performed during a radiotherapy treatment of the subject to generate real-time MRI images of the subject, thereby tracking the motion of internal organs of the subject and guiding the delivery of the radiotherapy treatment on the subject. Specifically, before a radiotherapy treatment is performed, the processing device 120 may instruct an MRI device (e.g., the MRI scanner 111 in FIG. 1) to start an MRI scan of the target subject. The MRI scan may continue throughout the radiotherapy process to monitor the state of the target subject in real time. The first imaging phase refers to an imaging phase before (i.e., before the start of the radiotherapy treatment) the radiotherapy device 112 emits the radiotherapy rays. The second imaging phase refers an imaging phase after the radiotherapy device 112 emits the radiotherapy rays (i.e., after the start of the radiotherapy treatment).

It should be noted that the above description regarding the process 300 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 300 may be accomplished with one or more additional operations not described and/or without one or more of the operations discussed above. For example, the process 300 may include an additional operation to transmit the real-time MRI images to a terminal device (e.g., a terminal 140 of a doctor) for display.

FIG. 5 is a flowchart illustrating an exemplary process 500 for determining a temporal factor φᵢ according to some embodiments of the present disclosure. In some embodiments, at least a portion of the process 500 may be performed to achieve operation 304 as described in connection with FIG. 3.

In 501, the processing device 120 (e.g., the determination module 201) may determine a transformation coefficient T based on the first target MRI signals.

As described elsewhere in this disclosure, the transformation coefficient T may represent a relationship between auxiliary signals and temporal factors. In some embodiments, the processing device 120 may determine the transformation coefficient T based on the first target auxiliary signals in a similar manner as described in operation 301.

In 502, the processing device 120 (e.g., the generation module 202) may determine the temporal factor q; based on the transformation coefficient T and the second target MRI signals Mᵢ.

As described elsewhere in this disclosure, the second target MRI signals Mᵢ may include a plurality of second target auxiliary signals collected at time Tᵢ. The processing device 120 may determine the temporal factor φᵢ based on the transformation coefficient T and the plurality of second target auxiliary signals of the second target MRI signals Mᵢ.

In some embodiments, the processing device 120 fill the plurality of second target auxiliary signals into the K-space to obtain a second K-space matrix. The processing device 120 determine the temporal factor φᵢ based on the transformation coefficient T and the second K-space matrix. For example, the processing device 120 may determine the temporal factor φᵢ according to Equation (6) as below:

$${φ}_{i} = {D}^{- 1} {U}_{k}^{H} {K}_{2} , \qquad ( 6 )$$

where, K₂ denotes the second K-space matrix,

$${D}^{- 1} {U}_{k}^{H}$$

denotes the transformation coefficient T determined in the operation 501.

In some embodiments, the processing device 120 may update the value of the transformation coefficient T based on the first target auxiliary signals and a plurality of reference auxiliary signals collected after the first target auxiliary signals (e.g., at least a portion of the second target auxiliary signals). For example, the processing device 120 may perform SVD on the first target auxiliary signals and the at least a portion of the second target auxiliary signals to determine the updated transformation coefficientT*. Since the updated transformation coefficient T* is determined based on more auxiliary signals than the original transformation coefficient T, the updated transformation coefficient T* may have an improved accuracy, thereby improving the accuracy of the generated real-time MRI image Aᵢ.

The process device 120 may determine the temporal factor φᵢ based on the plurality of second target auxiliary signals and the updated transformation coefficient T*. For example, the processing device 120 may determine the temporal factor φᵢ according to Equation (7) as below:

$${φ}_{i} = {( {D}^{- 1} {U}_{k}^{H} )}^{*} {K}_{2} , \qquad ( 7 )$$

where K₂ denotes the second K-space matrix,

$$( {D}^{- 1} {U}_{k}^{H} )$$

donates the updated transformation coefficient T*.

FIG. 6 is a flowchart illustrating an exemplary process 600 for determining a spatial factor U; according to some embodiments of the present disclosure. In some embodiments, at least a portion of the process 600 may be performed to achieve operation 304 as described in connection with FIG. 3.

In 601, the processing device 120 (e.g., the determination module 201) may determine, based on the second target MRI signals {M₁, M₂, . . . , Mₜ}, a second temporal factor φ′ⱼ, Mₜ being the latest second target MRI signals obtained before the determination of the spatial factor Uⱼ.

In some embodiments, the processing device 120 may determine the second temporal factor φ′ⱼ in a similar manner as how the temporal factor φᵢ is determined described in FIG. 5. For example, the processing device 120 fill the second target auxiliary signals of the second target MRI signals {M₁, M₂, . . . , Mₜ} into the K-space to obtain a third K-space matrix. In some embodiments, the processing device 120 fill the second target auxiliary signals of the second target MRI signals {M₁, M₂, . . . , Mₜ} and at least a portion of the first target auxiliary signals into the K-space to obtain the third K-space matrix. The processing device 120 determine the second temporal factor φ′ⱼ based on the transformation coefficient T and the third K-space matrix. Alternatively, the processing device 120 may update the value of the transformation coefficient T based on the first target auxiliary signals and at least a portion of the second target auxiliary signals of the second target MRI signals {M₁, M₂, . . . , Mₜ}. The processing device 120 further determine the second temporal factor φ′ⱼ based on the updated transformation coefficient and the third K-space matrix.

In 602, the processing device 120 (e.g., the determination module 201) may determining, based on the second temporal factor φ′ⱼ and the second target MRI signals {Mₜ₋₁, . . . , Mₜ}, the spatial factor Uⱼ.

In some embodiments, the processing device 120 may determine a reference spatial factor based on the second temporal factor φ′ⱼ and the second target MRI signals {Mₜ₋₁, . . . , Mₜ}, Mₜ₋₁ being the latest second target MRI signals obtained before the determination of the spatial factor Uⱼ₋₁. As described elsewhere in this disclosure, the second target MRI signals may include the plurality of second target imaging signals. The processing device 120 may determine the reference spatial factor based on the second temporal factor φ′ⱼ and the second target imaging signals of the second target MRI signals {Mₜ₋₁, . . . , Mₜ}. For example, the reference spatial factor may be determined by solving a third optimization function like Equation (2) or Equation (3) discussed above. In the third optimization function, di may represent K-space data corresponding to the second target imaging signals {Mₜ₋₁, . . . , Mₜ} collected by the jth coil channel, Φ₀ may be replaced by the temporal factor φ′ⱼ, and other coefficients may be the same as those in Equation (2) or (3). The processing device 120 may then determine the spatial factor U; based on the reference spatial factor and the prior determined spatial factor Uⱼ₋₁. For example, the processing device 120 may determine a sum of the reference spatial factor and the spatial factor Uⱼ₋₁ as the spatial factor Uⱼ.

In some embodiments, since that the second imaging stage is relatively short and the amount of the collected second target imaging signals is relatively small, the reference spatial factor determined based on the second target imaging signals may have a limited accuracy. The processing device 120 may determine the reference spatial factor based on the second target imaging signals and other reference imaging signals collected before the second target imaging signals. For example, the reference imaging signals may include a portion of the first target imaging signals. In order to reduce the calculation time of the reference spatial factor (e.g., the time for solving the third optimization function), only a small amount of reference imaging signals may be used on the premise of ensuring the accuracy of the determined reference spatial factor.

In some embodiments, the processing device 120 may determine the spatial factor Uᵢ without determining the reference spatial factor. For example, the spatial factor Uⱼ may be determined by solving a fourth optimization function like Equation (2) or Equation (3) discussed above. In the fourth optimization function, di may represent K-space data corresponding to the first target imaging signals and the second target MRI signals {M₁, M₂, . . . , Mₜ} collected by the jth coil channel, do may be replaced by the temporal factor φ′ᵢ, and other coefficients may be the same as those in Equation (2) or (3). Compared with the third optimization function, the fourth optimization function is constructed based on the original first target imaging signals and the second target imaging signals of the second target MRI signals {M₁, M₂, . . . , Mₜ}, and solving the fourth optimization function may need more calculation resources. By determining the reference spatial factor and adding it to the prior determined spatial factor Uⱼ₋₁, instead of directly determining the spatial factor Uⱼ, the updating efficiency of the spatial factor U may be improved and the calculation resources may be saved, which, in turn, improves the real-time performance of imaging.

In some embodiments, the processing device 120 may update coil sensitivity maps of coils for collecting the first target MRI signals and the second target MRI signals based on the plurality of second target imaging signals of the second target MRI signals {M₁, M₂, . . . , Mₜ}. The processing device 120 may further determine the spatial factor Uⱼ based on the second temporal factor φ′ⱼ, the plurality of second target imaging signals of the second target MRI signals {M₁, M₂, . . . , Mₜ}, and the updated coil sensitivity maps. For example, in the third or fourth optimization function as aforementioned, Sⱼ may denote the updated coil sensitive map corresponding to the jth coil channel. A coil sensitivity map of a coil may reflect a distribution of the response degree of the coil with respect to different portions of the subject (i.e., the capacity for receiving MRI signals from different portions of the subject). Merely by way of example, for each of the coils, the processing device 120 may generate a coil image based on the second target imaging signals collected by the coil. The processing device 120 may then determine the updated coil sensitivity maps of different coils based on the coil images. In some embodiments, the updated coil sensitivity maps may be determined based on the combination of the first target imaging signals and the second target imaging signals.

According to some embodiments of the present disclosure, the temporal factors {φ₁, φ₂, . . . , φₙ} may be updated based on the plurality of second target auxiliary signals and the transformation coefficient T, and further one or more real-time MRI images of the subject may be generated based on the temporal factors {φ₁, φ₂, . . . , φₙ} and the initial spatial factor U₀. Because the value of the temporal factors may be updated based on a small amount of data, moreover, in some embodiments, the transformation coefficient T can be determined in advance, e.g., before the first second imaging sub-stage ends, the real-time MRI images of the second imaging stage may be generated with simple calculation (e.g., matrix multiplication and/or matrix division). In this way, the efficiency of the generation of the real-time MRI images of the subject can be improved, and real-time imaging with low latency (e.g., close to zero seconds) can be achieved. In cases that the motion magnitude of the subject from the first imaging stage to the second imaging stage is relatively small or the subject doesn't have motion, the generated real-time MRI images of the subject may have a desired accuracy and satisfy use requirements.

According to some embodiments of the present disclosure, the real-time MRI images of the subject may be generated based on based on the temporal factors {φ₁, φ₂, . . . , φₙ} and the spatial factors {U₁, U₂, . . . , Uₘ}. By monitoring the body motion of the subject and determining the spatial factors {U₁, U₂, . . . , Uₘ} when the subject has an obvious motion, the imaging quality of the generated real-time MRI images may be improved. In addition, the value of a spatial factor may be determined by determining the reference spatial factor and adding it to the prior spatial factor, which may improve the efficiency of the determination of the spatial factor. In this way, the efficiency of the generation of the real-time MRI images of the subject can be improved, and real-time imaging with low latency (e.g., smaller than 100 milliseconds) can be achieved.

In addition, the obvious motion of the subject may also result in changes in the coil sensitivity maps of coils. According to some embodiments of the present disclosure, the coil sensitivity maps of the coils may be updated if obvious motion of the subject is detected, and the values of the spatial factors may be determined based on the plurality of second target imaging signals and the updated coil sensitivity maps. In this way, the real-time MRI images of the subject obtained based on the temporal factors and the spatial factors may have a desire accuracy.

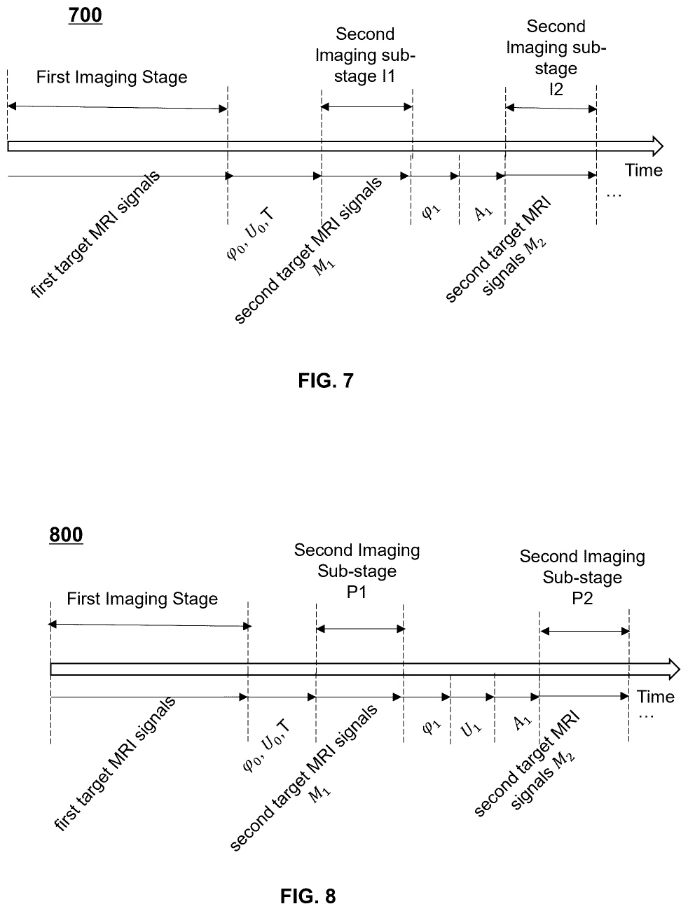

FIG. 7 is a schematic diagram illustrating an exemplary process 700 for generating a real-time MRI image of a subject according to some embodiments of the present disclosure. As shown in FIG. 7, an MRI scan of the subject may include a first imaging stage and a plurality of second imaging sub-stages after the first imaging stage (e.g., second imaging sub-stages 11 and 12 as shown in FIG. 7). In the first imaging stage, first target MRI signals may be collected. Then, an initial temporal factor φ₀, a transformation coefficient T, and an initial spatial basis function U₀ may be determined based on the collected first target MRI signals. In each of the plurality of second imaging sub-stages, second target MRI signals may be collected. In some embodiments, the initial temporal factor φ₀, the transformation coefficient T, and the initial spatial factor U₀ may be determined before the second imaging stage 11. Alternatively, a time period for determining the initial temporal factor φ₀, the transformation coefficient T, and the initial spatial factor U₀ may at least partially overlap with the second imaging stage 11. Then the temporal factor φ₁ may be determined based on the collected second target MRI signals M₁. Further, the real-time MRI image A₁ of the subject may be generated based on the temporal factor φ₁ and the spatial factor U₀.

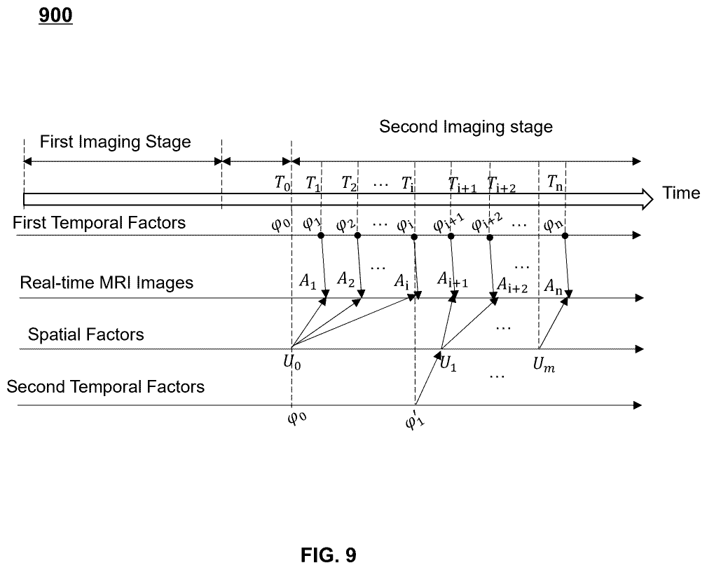

FIG. 8 is a schematic diagram illustrating an exemplary process 800 for generating a real-time MRI image of a subject according to some embodiments of the present disclosure. As shown in FIG. 8, an MRI scan of the subject may include a first imaging stage and a plurality of second imaging sub-stages after the first imaging stage (e.g., second imaging stages P1 and P2 as shown in FIG. 8). In the first imaging stage, first target MRI signals may be collected. Then, an initial temporal factor φ₀, a transformation coefficient T, and an initial spatial basis function U₀ may be determined based on the collected first target MRI signals. In each of the plurality of second imaging sub-stages, second target MRI signals may be collected. In some embodiments, the initial temporal factor φ₀, the transformation coefficient T, and the initial spatial factor U₀ may be determined before the second imaging stage P1. Alternatively, a time period for determining the initial temporal factor φ₀, the transformation coefficient T, and the initial spatial factor U₀ may at least partially overlap with the second imaging stage P1. Then the temporal factor φ₁ may be determined based on the collected second target MRI signals M₁. Further, the spatial factor U₁ may be determined based on the second target MRI signals M₁ and the first target MRI signals. Finally, the real-time MRI image A₁ of the subject may be generated based on the temporal factor φ₁ and the spatial factor U₁.

FIG. 9 is a schematic diagram illustrating an exemplary process 900 for generating real-time MRI images of a subject according to some embodiments of the present disclosure. As shown in FIG. 9, an MRI scan of the subject may include a first imaging stage and a second imaging stage after the first imaging stage. In the first imaging stage, first target MRI signals may be collected. Then, an initial temporal factor do and an initial spatial basis function U₀ may be determined based on the collected first target MRI signals.

In the second imaging stage, second target MRI signals {M₁, M₂, . . . , Mₙ} may be collected at each time among time series {T₁, T₂, . . . , Tₙ}. First temporal factors {φ₁, φ₂, . . . , φₙ} and spatial factors {U₁, U₂, . . . , Uₘ} may be determined based on the second target MRI signals {M₁, M₂, . . . , Mₙ}, wherein m is smaller than or equal to n. Specifically, each time second target MRI signals are collected at one time among time series {T₁, T₂, . . . , Tₙ}, a corresponding first temporal factor may be determined based on the second target MRI signals collected at the time. For example, after the second target MRI signals Mᵢ are collected at time T₁, the first temporal factor φᵢ may be determined based on the second target MRI signals Mᵢ.

Each of the spatial factors {U₁, U₂, . . . , Uₘ} may be determined based on the second target MRI signals obtained before the determination of the spatial factor. For example, the spatial factor U₁ may be determined based on the second target MRI signals {M₁, M₂, . . . , Mᵢ}. In some embodiments, for the spatial factor Uⱼ, a second temporal factor φ′ⱼ may be determined based on the second target MRI signals obtained before the determination of the corresponding spatial factor Uⱼ. Then, the spatial factor Uⱼ may be determined based on the corresponding second temporal factor φ′ⱼ. For example, as show in FIG. 9, the second temporal factor φ′₁ may be determined based on the second target MRI signals {M₁, M₂, . . . , Mᵢ}. The spatial factor U₁ may be determined based on the second temporal factor φ′₁.

Real-time MRI images {A₁, A₂, . . . , Aₙ} may be generated during the process of the second imaging stage. A real-time MRI image may reflect the status of the subject at one time of the time series {T₁, T₂, . . . , Tₙ}. For example, Aᵢ may reflect the status of the subject at the time Tᵢ. Each of the real-time MRI images {A₁, A₂, . . . , Aₙ} may be generated based on the latest determined temporal factor and one of the initial spatial factor U₀ or the latest determined spatial factor. For example, as show in FIG. 9, the real-time MRI image Aᵢ may be generated based on the temporal factor φᵢ and the initial spatial factor U₀. As another example, the real-time MRI image Aₜ₊₁ may be generated based on the temporal factor φᵢ₊₁ and the latest determined spatial factor U₁.

In some embodiments, the process 900 may be performed by the processing device 120 using a plurality of threads. For example, the process 900 may be performed using a first thread and a second thread. The first thread and a second thread may be operated simultaneously. In some embodiments, the processing device 120 may obtain MRI signals (e.g., the first target MRI signals, the second target MRI signals {M₁, M₂, . . . , Mₙ}), determine temporal factors (e.g., the initial temporal factors φ₀, the temporal factors {φ₁, φ₂, . . . , φₙ}), and generate real-time MRI images (e.g., the real-time MRI images {A₁, A₂, . . . , Aₙ}) using the first thread. The processing device 120 may determine spatial factors (e.g., the initial spatial factor U₀, the spatial factors {U₁, U₂, . . . , Uₘ}) using the second thread. The second thread may be further configured to feed the determined spatial factors to the first thread.

For example, the obtaining of the second target MRI signals {M₁, M₂, . . . , Mₙ}, the determination of the first temporal factors {φ₁, φ₂, . . . , On}, and the generation of the real-time MRI images {A₁, A₂, . . . , Aₙ} may be implemented by the first thread. The determination of the spatial factors {U₁, U₂, . . . , Uₘ} may be implemented by the second thread. The second thread may be further configured to feed the determined spatial factors {U₁, U₂, . . . , Uₘ} to the first thread. In some embodiments, the first thread may be further configured to determine the second temporal factor {φ′₁, φ′₂, . . . , φ′ₘ}, and feed the determined the second temporal factors {φ′₁, φ′₂, . . . , φ′ₘ} to the second thread. The spatial factor U; may be determined by the second thread based on the second target MRI signals {M₁, M₂, . . . , Mₜ} and the second temporal factor φ′ⱼ.

The determinations of a temporal factor and a real-time MRI image involve a relatively small amount of computation (e.g., only the computation of the corresponding auxiliary signals), which may have a relatively high efficiency and require a relatively smaller amount of computational resources. However, the determination of each spatial factor involves a relatively large amount of computation (e.g., the computation of the corresponding auxiliary signals, imaging signals, etc.), which may need more computational resources. The first thread for determining the temporal factors and real-time MRI images and the second thread for determining the spatial factors may be operated independently, which may ensure that the calculation of the temporal factors and real-time MRI images is not affected by the calculation of the spatial factors, thereby achieving real-time imaging. In some embodiments, a specific resource allocation strategy may be adopted to preferentially execute the first thread. If the computing resources are sufficient, the second thread may be performed. If there are insufficient computing resources, only the first thread is executed. In this way, the continuity of real-time imaging may be ensured, so that users will not experience jamming when viewing the real-time MRI images.

FIG. 10 is a flowchart illustrating an exemplary process for MRI-guided radiotherapy according to some embodiments of the present disclosure. In some embodiments, a process 1000 in FIG. 10 may be implemented to detect a motion of a target subject based on MRI signals (auxiliary signals and imaging signals) collected in an MRI scan, and generate a real-time image of the target subject to achieve radiotherapy guidance. In some embodiments, the process 1000 may be implemented by the processing device 140 or one or more modules shown in FIG. 2. For example, the process 1000 may be implemented by or one or more modules of the processing device 140. As shown in FIG. 10, the process 1000 may include the following operations.

In 1001, the processing device 120 (e.g., the acquisition module 202) may obtain first auxiliary signals collected by an MRI device during an MRI scan of a target subject.

As described above, before the radiotherapy device 112 emits radiotherapy rays, the MRI device (e.g., the MRI scanner 111 in FIG. 1) may collect auxiliary signals and imaging signals by performing a first imaging phase. The first auxiliary signals may be collected by the MRI device at a first time before the radiotherapy rays are emitted. The first auxiliary signals may be used as reference signals for detecting the motion of the target subject. In other words, the first auxiliary signals are auxiliary signals collected by the MRI device at the first time before the radiotherapy device 112 emits the radiotherapy rays. The first time may be any time period or time point in the first imaging phase described above. In other words, the first auxiliary signals may include any one or more auxiliary signals collected in the first imaging phase (i.e., before the radiotherapy rays are emitted). For example, the first auxiliary signals be last auxiliary signals collected in the first imaging phase.

In some embodiments, the first imaging stage may include a breath-holding period for the target subject to hold breath. The first time may be within the breath-holding period. In some embodiments, the target subject may maintain normal breathing during the first imaging phase. The first time is a period or time point during which the target subject maintains normal breathing.

In 1002, the processing device 120 (e.g., the acquisition module 202) may obtain second auxiliary signals collected by the MRI device during the MRI scan of the target subject.

The second auxiliary signals may be collected by the MRI device at a second time after the radiotherapy rays are emitted. In other words, the second auxiliary signals may be auxiliary signals collected by the MRI device at the second time after the radiotherapy device 112 emits the radiotherapy rays. The second time may be any time period or time point later than the first time in the second imaging phase described above. That is to say, the second auxiliary signals may be any one or more auxiliary signals collected in the second imaging phase (i.e., after the radiotherapy rays are emitted). In some embodiments, whenever the MRI device collects new auxiliary signals at a certain time in the second imaging phase, the processing device 140 may use the auxiliary signals as the second auxiliary signals, and determine whether the target subject has an obvious motion (e.g., a motion with an amplitude greater than a threshold) at the time based on the first auxiliary signals and the second auxiliary signals.

In 1003, the processing device 140 (e.g., the determination module 201) may determine whether a first motion amplitude of the target subject from the first time to the second time is greater than a threshold based on the first auxiliary signals and the second auxiliary signals.

As used herein, a motion amplitude of the target subject refers to a motion amplitude or a motion displacement of the target subject.

In some embodiments, the first motion amplitude may be represented by a difference between the first auxiliary signals and the second auxiliary signals. The difference between the first auxiliary signals and the second auxiliary signals may be determined based on k-space data or image domain data. Accordingly, the threshold refers to a threshold corresponding to the difference between the first auxiliary signals and the second auxiliary signals. In some embodiments, when the first auxiliary signals include multiple auxiliary signals and/or the second auxiliary signals include multiple auxiliary signals, the difference between the first auxiliary signals and the second auxiliary signals may be determined based on an average value of the first auxiliary signals and/or an average value of the second auxiliary signals.

When the difference between the first auxiliary signals and the second auxiliary signals is smaller than or equal to the threshold, the processing device 140 may determine that the first motion amplitude of the target subject from the first time to the second time is smaller than or equal to the threshold. When it is determined that the difference between the first auxiliary signals and the second auxiliary signals is greater than the threshold, the processing device 140 may determine that the first motion amplitude of the target subject from the first time to the second time is greater than the threshold.

Since the auxiliary signals are projections of imaging data of the target subject in a certain direction and capable of reflecting the occurrence of a motion (e.g., an overall body motion), the motion of the target subject can be effectively detected based on the difference between the first auxiliary signals and the second auxiliary signals.

In some embodiments, as described above, the auxiliary signals have a preset readout direction. When the readout direction of the auxiliary signals is substantially parallel to a motion direction of a target region, the auxiliary signals can accurately reflect the motion of the target region. However, the motion of some target regions (e.g., a heart) do not have a clear motion direction (different points on the heart have different motion directions), and the auxiliary signals with the preset readout direction cannot accurately detect the motion of the target region. In order to more accurately detect the motion of the target region, in addition to the auxiliary signals, the MRI device may also be instructed to collect reference auxiliary signals during the MRI scan. The auxiliary signals may be used for motion detection and MRI imaging simultaneously, while the reference auxiliary signals may be only used for motion detection. The reference auxiliary signals may be collected in a similar manner to the auxiliary signals, but a readout direction of the reference auxiliary signals may be different from the readout direction of the auxiliary signals. For the convenience of description, the readout direction of the auxiliary signals (including the first auxiliary signals and the second auxiliary signals) may be recorded as a first readout direction. The readout direction of the reference auxiliary signals may be recorded as a second readout direction. The second readout direction may be different from the first readout direction. For example, the second readout direction may be perpendicular to the first readout direction. Merely by way of example, if the target region includes the heart, the first readout direction may be a head-to-foot direction of the target subject, and the second readout direction may be a front-to-back direction of the target subject.

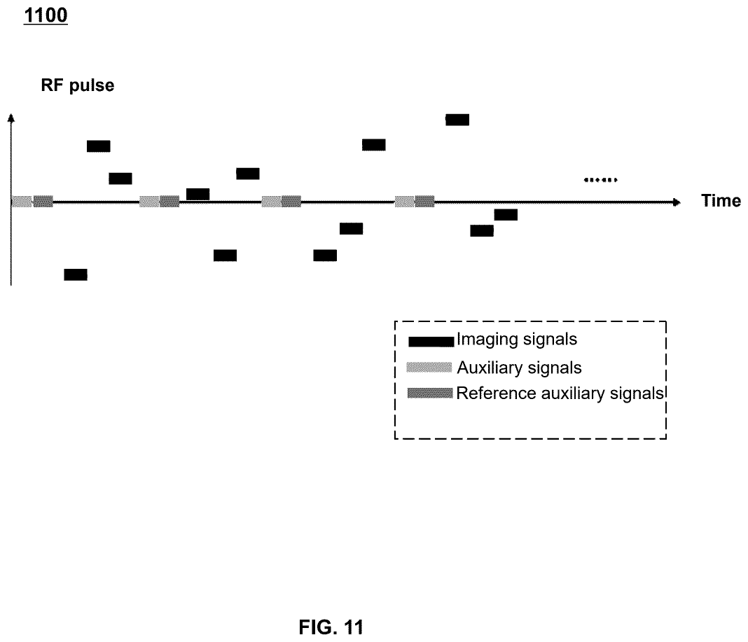

Merely by way of example, FIG. 11 is a schematic diagram illustrating an exemplary MRI pulse sequence for implementing an MRI scan on a target subject according to some embodiments of the present disclosure. As shown in FIG. 11, after one auxiliary signal for imaging is collected, one reference auxiliary signal and three imaging signals may be collected.

When motion detection is performed, the processing device 140 may determine whether the first motion amplitude of the target subject is greater than the threshold based on the first auxiliary signals, the second auxiliary signals, and the reference auxiliary signals. For example, the processing device 140 may obtain first reference auxiliary signals and second reference auxiliary signals collected by the MRI device. The first reference auxiliary signals may be collected after the first time, and the second reference auxiliary signals may be collected after the second time. The first reference auxiliary signals and the second reference auxiliary signals may have a second readout direction. The second readout direction may be different from the first readout direction. For example, the second readout direction may be perpendicular to the first readout direction. It should be noted that a time difference between a collection time of the first reference auxiliary signals and the first time may be relatively small (smaller than the threshold), so that the first reference auxiliary signals may approximately reflect a state of the target subject at the first time. A time difference between a collection time of the second reference auxiliary signals and the second time may be relatively small (smaller than the threshold), so that the second reference auxiliary signals may approximately reflect a state of the target subject at the second time. For example, the MRI device may collect the first reference auxiliary signals immediately after the first auxiliary signals are collected, and collect the second reference auxiliary signals immediately after the second auxiliary signals are collected.

Further, the processing device may determine whether the first motion amplitude of the target subject from the first time to the second time is greater than the threshold based on the first auxiliary signals, the second auxiliary signals, the first reference auxiliary signals, and the second reference auxiliary signals. In this case, the first motion amplitude may be represented by a first difference between the first auxiliary signals and the second auxiliary signals and a second difference between the first reference auxiliary signals and the second reference auxiliary signals. When both the first difference and the second difference are smaller than or equal to the threshold, the processing device 140 may determine that the first motion amplitude of the target subject from the first time to the second time is smaller than or equal to the threshold. When it is determined that any one of the first difference and the second difference is greater than the threshold, the processing device 140 may determine that the first motion amplitude of the target subject from the first time to the second time is greater than the threshold.

By using the auxiliary signals and the reference auxiliary signals with different readout directions, the accuracy of motion detection can be improved, thereby improving the accuracy of the guided radiotherapy.

In 1004, the processing device 140 (e.g., the determination module 201) may determine whether to stop emitting the radiotherapy rays based on a determination result of whether the first motion amplitude is greater than the threshold.

In some embodiments, in response to determining that the first motion amplitude is smaller than or equal to the threshold, the processing device 140 may determine to continue emitting the radiotherapy rays and generate a first MRI image corresponding to the second time. Descriptions regarding the generation of the first MRI image may be found in FIG. 12 and related descriptions thereof, which are not repeated here.

In some embodiments, in response to determining that the first motion amplitude is greater than the threshold, the processing device 140 may instruct the radiotherapy device to stop emitting the radiotherapy rays, and continue to monitor the motion of the target subject based on new auxiliary signals collected during the MRI scan. For example, the processing device 140 may obtain third auxiliary signals collected by the MRI device at third time. The third time may be later than the second time. The third auxiliary signals may be any auxiliary signals collected later than the second auxiliary signals. Merely by way of example, after stop emitting the radiotherapy rays is determined, each time the MRI device collects one auxiliary signal, the processing device 140 may use the auxiliary signal as the third auxiliary signal. Furthermore, the processing device 140 may further determine whether to continue emitting the radiotherapy rays based on the third auxiliary signals.

Specifically, the processing device 140 may determine whether a second motion amplitude of the target subject from the first time to the third time is greater than or equal to the threshold based on the first auxiliary signals and the third auxiliary signals. In some embodiments, the determination of whether the second motion amplitude is greater than or equal to the threshold may be performed in a similar manner as that of whether the first motion amplitude is greater than the threshold. For example, the second motion amplitude may be represented by a difference between the first auxiliary signals and the third auxiliary signals. As another example, the processing device 140 may determine whether the second motion amplitude of the target subject is greater than the threshold based on the first auxiliary signals, the third auxiliary signals, and the reference auxiliary signals. In response to determining that the second motion amplitude is smaller than or equal to the threshold, the processing device 140 may instruct the radiotherapy device to continue emitting the radiotherapy rays and generate the second MRI image corresponding to the third time in real time. Description regarding the generation of the second MRI image may be found in FIG. 13 and related descriptions thereof, which are not repeated here. When the second motion amplitude is smaller than or equal to the threshold, it means that the target subject returns to a position close to the position at the first time. In this case, the radiotherapy may continue. By continuously detecting the motion of the target subject and restarting radiotherapy when the motion amplitude of the target subject falls below the threshold, the time of interrupting radiotherapy can be reduced, and the efficiency of radiotherapy can be improved.

In some embodiments, in response to determining that the second motion amplitude is greater than the threshold, the processing device 140 may determine whether a third motion amplitude of the target subject from the second time to the third time is greater than the threshold based on the second auxiliary signals and the third auxiliary signals. In some embodiments, the determination of whether the third motion amplitude is greater than the threshold may be performed in a similar manner as that of whether the first motion amplitude is greater than the threshold. For example, the third motion amplitude may be represented by a difference between the second auxiliary signals and the third auxiliary signals. As another example, the processing device 140 may determine whether the third motion amplitude of the target subject is greater than the threshold based on the second auxiliary signals, the third auxiliary signals, and the reference auxiliary signals. In response to determining that the third motion amplitude is smaller than or equal to the threshold, the processing device 140 may instruct the radiotherapy device to continue emitting the radiotherapy rays and generate a third MRI image corresponding to the third time in real time. Description regarding the generation of the third MRI image may be found in FIG. 14 and related descriptions thereof, which are not repeated here. When the second motion amplitude is greater than the threshold and the third motion amplitude is smaller than or equal to the threshold, it means that although the target subject does not return to the position close to the position at the first time, the target subject maintains at a position at the second time and does not continue to move. In this case, the radiotherapy can continue. In this way, the time of interrupting radiotherapy can be reduced and the efficiency of radiotherapy can be improved.

In some embodiments, only when the time difference between the second time and the third time exceeds a threshold time and the third motion amplitude is smaller than or equal to the threshold (i.e., when the target subject maintains at the position at the second time for a certain time), the processing device 140 may instruct the radiotherapy device to continue emitting the radiotherapy rays. In some embodiments, when the target subject maintains at the position at the second time for a certain time, the processing device 140 may determine whether a data volume of MRI signals (including the auxiliary signals and the imaging signals) collected from the second time to the third time is sufficient to update a temporal factor and/or a spatial factor. When the data volume is sufficient, the processing device 140 may use a time period between the second time and the third time as a new first imaging phase, auxiliary signals (e.g., the second auxiliary signals, the third auxiliary signals, etc.) in the new first imaging phase as new first auxiliary signals (reference signals for motion detection), and certain auxiliary signals collected after the third auxiliary signals are collected as new second auxiliary signals. The processing device 140 may repeat the process 1000 to continue monitoring the motion of the target subject.

In some embodiments, the processing device 140 may determine position information of a region of interest (ROI) of the target subject at the third time based on the third MRI image, and update radiation parameters of the radiotherapy rays based on the position information of the ROI at the third time. Exemplary radiation parameters may include a radiation angle, a dose distribution, a radiation intensity, etc. In some embodiments, the processing device 140 may instruct the radiotherapy device to continue emitting the radiotherapy rays based on updated radiation parameters. The accuracy of radiotherapy can be improved by adjusting the radiation parameters in time.

In some embodiments, in response to determining that both the second motion amplitude and the third motion amplitude are greater than the threshold, it means that the target subject neither returns to the position at the first time nor maintains at the position at the second time. The processing device 140 may determine that it is necessary to continue to interrupt the emission of radiotherapy rays, and issue a prompt message to remind the target subject to stop moving. After the target subject stops moving for more than a certain time, the processing device 140 may determine new radiation parameters based on the latest position information of the target subject, and instruct the radiotherapy device to continue to emit radiotherapy rays. In some embodiments, when the MRI device collects new auxiliary signals at a new time, the processing device 140 may use it as new second auxiliary signals, and determine whether the first motion amplitude of the target subject is smaller than or equal to the threshold (i.e., whether it returns to the position at the first time) based on the first auxiliary signals and the new second auxiliary signals, or determine whether the second motion amplitude of the target subject is smaller than or equal to the threshold (i.e., whether it maintains at the new position) based on the new second auxiliary signals and subsequently collected third auxiliary signals. Only when the target subject returns to the position at the first time, or the target subject maintains at the new position for a certain period of time, the processing device 140 may determine that it is necessary to continue to emit radiotherapy rays.

According to the process 1000, systems and methods of the present disclosure may detect the motion of the target subject based on the auxiliary signals collected by the MRI device without relying on an additional motion detection device, thereby reducing cost. Moreover, since the auxiliary signals can accurately reflect the motion of the target subject, systems and methods of the present disclosure can obtain accurate motion detection results based on the auxiliary signals, thereby improving the accuracy of the guided radiotherapy. Furthermore, since the collection speed of the auxiliary signals is fast and computational complexity of determining motion detection results based on the auxiliary signals is small, systems and methods of the present disclosure can quickly obtain the motion detection results based on the auxiliary signals, thereby improving the efficiency of the guided radiotherapy.

In some embodiments, the motion of the target subject is continuously detected based on the auxiliary signals, and accurate MRI images can be quickly generated for different motion detection results, so as to make timely adjustments to the radiotherapy process, thereby improving the efficiency and accuracy of the radiotherapy process, and even achieving real-time guided radiotherapy.

It should be noted that the above description of the process 1000 is only for example and illustration, and does not limit the scope of application of the present disclosure. For those skilled in the art, various modifications and changes can be made to the process 1000 under the guidance of the present disclosure. However, these modifications and changes are still within the scope of the present disclosure. In some embodiments, the process 1000 may be completed by one or more additional steps not described and/or without one or more of the above steps. For example, the process 1000 may include an additional step of transmitting the generated MRI images (e.g., the first MRI image, the second MRI image, the third MRI image, etc.) to a terminal device (e.g., a doctor terminal 130) for display.

In some embodiments, the motion of the target subject may be detected using other manners to determine the motion amplitude of the target subject. For example, the motion of the target subject may be detected using an additional motion detection device (e.g., an optical camera, a radar device, etc.). The additional motion detection device may be also referred to as the image acquisition device in FIG. 3, and more descriptions regarding the detection of the motion and/or the addition motion device may be found in FIG. 3, which are not repeated here.

FIG. 12 is a flowchart illustrating an exemplary process for generating a first MRI image during MRI-guided radiotherapy according to some embodiments of the present disclosure. In some embodiments, when it is determined that the first motion amplitude from the first time to the second time is smaller than the threshold, a process 1200 may be implemented. The process 1200 may be implemented by the processing device 120 or one or more modules shown in FIG. 2. For example, the process 1200 may be implemented by the determination module 201 and the generation module 203 of the processing device 120. As shown in FIG. 12, the process 1200 may include the following operations.

In 1201, the processing device 120 (e.g., the determination module 201) may obtain the first imaging signals collected by an MRI device before radiotherapy rays are emitted.

As described above, before the radiotherapy device 112 emits the radiotherapy rays, the MRI device may collect auxiliary signals and imaging signals by performing a first imaging phase. The imaging signals collected in the first imaging phase may be first imaging signals (i.e., the first target imaging signals). The processing device 120 may obtain the first imaging signals from the MRI device or a storage device (e.g., the storage device 130) configured to store MRI signals.

In 1202, the processing device 120 (e.g., the determination module 201) may determine an initial temporal factor and an initial spatial factor based on first auxiliary signals and the first imaging signals. In some embodiments, the operation 1202 may be performed simultaneously with or before the operation 1002.

In some embodiments, the operation 1202 may be similar to the operation 303, and related descriptions may be found in FIG. 3.

In some embodiments, the processing device 120 may determine a transformation coefficient and the initial temporal factor based on the first auxiliary signals. The transformation coefficient may represent a relationship between auxiliary signals and temporal factors. In some embodiments, the plurality of first auxiliary signals may be filled into K-space to obtain a fourth K-space matrix. The processing device 120 may determine the transformation coefficient and the initial temporal factor based on the fourth K-space matrix. For example, the processing device 120 may determine the transformation coefficient and the initial temporal factor according to a singular value decomposition (SVD) algorithm. Merely by way of example, the fourth K-space matrix may be denoted as K₄.

The fourth K-space matrix K₄ may be presented as

$$( \begin{matrix}κ ( {k}_{1} , {t}_{1} ) & … & κ ( {k}_{1} , {t}_{N} ) \\ κ ( {k}_{2} , {t}_{1} ) & … & κ ( {k}_{2} , {t}_{N} ) \\ ⋮ & … & ⋮ \\ κ ( {k}_{c} , {t}_{1} ) & … & κ ( {k}_{c} , {t}_{N} )\end{matrix} ) ,$$

wherein an element in the fourth K-space matrix K₄ represents k-space data collected by a specific coil channel at a certain moment. For example, κ(k_{c}, t₁) represents k-space data collected by the coil channel k_{c} at a moment t₁. The processing device 120 may determine the transformation coefficient and the initial temporal factor by performing the SVD on the fourth K-space matrix K₄ according to Equation (8) as below:

$${K}_{4} = {U}_{k} D {φ}_{0} , \qquad ( 8 )$$

where Uₖ denotes a projection coefficient matrix, φ₀ denotes the initial temporal factor (e.g., in the form of at least one temporal factor matrix), D denotes a singular value matrix. The transformation coefficient T may be determined based on the projection coefficient matrix Uₖ and the singular value matrix D. For example, the transformation coefficient T may be (UₖD) or

$$( {D}^{- 1} {U}_{k}^{H} )$$

(in which

$${U}_{k}^{H}$$

donates a conjugate transpose matrix of the projection coefficient matrix Uₖ, and D⁻¹ denotes an inverse matrix of the singular value matrix D).

In some embodiments, the first auxiliary signals may only include a portion of auxiliary signals collected in the first imaging phase. The processing device 120 may determine the transformation coefficient T and the initial temporal factor do based on the first auxiliary signals and other auxiliary signals collected in the first imaging phase. The determination of the transformation coefficient T and the initial temporal factor φ₀ based on the first auxiliary signals and other auxiliary signals may be performed in a similar manner as that of the transformation coefficient T and the initial temporal factor do based on the first auxiliary signals described above.

Further, the processing device 120 may determine the initial spatial factor U₀ based on the initial temporal factor do and the first imaging signals. In some embodiments, the processing device 120 may construct an optimization function relating to the initial spatial factor U₀. The optimization function may incorporate the plurality of first imaging signals and the initial temporal factor φ₀. The processing device 120 may further determine the initial spatial factor U₀ by solving the optimization function. For example, the processing device 120 may determine the initial spatial factor U₀ of the subject according to a fifth optimization function shown in Equation (9) as below:

$$= {arg min}_{{U}_{0}} {\sum }_{ℏ = 1}^{c} {‖ Ω {FS}_{h} {U}_{0} {Φ}_{0} - {d}_{h} ‖}_{2}^{2} , \qquad ( 9 )$$

where [symbol] denotes the optimal spatial factor determined by solving Equation (9), U₀ denotes the initial spatial factor φf the target subject (e.g., in the form of at least one spatial factor matrix), c denotes the count of coil channels of the MRI scanner, dₕ denotes K-space data obtained by filling first imaging signals acquired by the hth coil channel into the K-space, Ω denotes an undersampling operator (which may be omitted in some conditions), F denotes a Fourier transformation operator, h denotes the number of the coil channel, Sₕ denotes a coil sensitive map corresponding to the h^{th} coil channel, Φ₀ denotes the initial temporal factor do of the target subject (e.g., in the form of at least one temporal factor matrix).

As another example, the processing device 120 may determine the initial spatial factor U₀ of the subject according to a sixth optimization function shown in Equation (10) as below:

$$= {arg min}_{{U}_{0}} {\sum }_{ℏ = 1}^{c} {‖ Ω {FS}_{h} {U}_{0} {Φ}_{0} - {d}_{h} ‖}_{2}^{2} + λ {‖ TV ( {U}_{0} ) ‖}_{1} , \qquad ( 10 )$$

where λ denotes a regularization parameter, TV(Uₜᵣ) denotes a 3-dimensional total variation, and λ∥TV(Utr)∥₁ denotes a constraint item relating to the at least one spatial factor matrix of the subject (which may be omitted in some conditions).

In 1203, in response to determining that the first motion amplitude is smaller than or equal to the threshold, the processing device 120 (e.g., the determination module 201) may determine a first updated temporal factor based on second auxiliary signals.

When the first motion amplitude is smaller than or equal to the threshold, it means that the target subject has no obvious motion from the first time to the second time, and only the temporal factor needs to be updated without updating the initial spatial factor U₀.

In some embodiments, the processing device 120 may determine the first updated temporal factor in a similar manner as how to determine the temporal factor φᵢ described in FIG. 5. For example, the processing device 120 may fill the second auxiliary signals into the K-space to obtain a fifth K-space matrix. Further, the processing device 120 may determine the first updated temporal factor by performing the SVD on the fifth K-space matrix in a similar manner as how to determine the initial temporal factor in the operation 1202.

As another example, the processing device 120 may determine the first updated temporal factor based on the fifth K-space matrix and the transformation coefficient T. For example, the processing device 120 may determine the first updated temporal factor according to Equation (11) as below:

$${φ}_{1} = {D}^{- 1} {U}_{k}^{H} {K}_{5} , \qquad ( 11 )$$

where, φ₁ denotes the first updated temporal factor, K₅ denotes the fifth K-space matrix, and

$${D}^{- 1} {U}_{k}^{H}$$

denotes the transformation coefficient T determined in the operation 1202.

In 1204, the processing device 120 (e.g., the generation module 203) may generate the first MRI image of the target subject based on the first updated temporal factor and the initial spatial factor. The first MRI image may correspond to the second time.

The first MRI image may reflect a state of the target subject at the second time. In some embodiments, the first MRI image may be a two-dimension (2D) image, a three-dimension (3D) image, or the like.

In some embodiments, the first MRI image of the target subject may be represented by a multi-dimensional tensor, which may be determined based on the first updated temporal factor φ₁ and the initial spatial factor U₀. For example, with the first updated temporal factor φ₁ and the initial spatial factor U₀ available, the processing device 120 may generate the first MRI image A₁ of the target subject by determining a product of at least one temporal factor matrix and at least one spatial factor matrix. The at least one temporal factor matrix may include the first updated temporal factor φ₁ and the at least one spatial factor matrix may include the initial spatial factor U₀. Merely by way of example, the processing device 120 may generate the first MRI image A₁ of the target subject according to Equation (12) as below:

$${A}_{1} = {U}_{0} {φ}_{1} , \qquad ( 12 )$$

where A₁ denotes a multi-dimensional tensor for representing the first MRI image A₁ of the target subject, φ₁ denotes the first updated temporal factor in the form of the at least one temporal factor matrix, and U₀ denotes the initial spatial factor in the form of the at least one spatial factor matrix.

In some embodiments, the processing device 120 may generate the first MRI image A₁ of the target subject corresponding to a certain time-varying dimension based on the temporal factor matrix corresponding to the certain time-varying dimension and the at least one spatial factor matrix. For example, the processing device 120 may generate the first MRI image A₁ of the heart by determining a product of at least one spatial factor matrix and the temporal factor matrix including the temporal factor relating to the cardiac motion. The temporal factor matrix relating to the cardiac motion may include the temporal factor relating to the cardiac motion.

According to the process 1200, when it is determined that the first motion amplitude is smaller than or equal to the threshold, the first MRI image may be generated based on the first updated temporal factor and the initial spatial factor. The first MRI image may be generated with simple calculation, and be output with low latency, thereby improving the efficiency of the radiotherapy. In some embodiments, the calculation of basic data can be performed immediately (e.g., before a second imaging phase or simultaneously with the collection of the MRI signals in radiotherapy) after the first auxiliary signals are collected. In this way, the delay in the generation of the first MRI image can be further reduced, thereby further improving the efficiency of the radiotherapy.

FIG. 13 is a flowchart illustrating an exemplary process for generating a second MRI image during MRI-guided radiotherapy according to some embodiments of the present disclosure. In some embodiments, when it is determined that the first motion amplitude from the first time to the second time is greater than the threshold and the second motion amplitude from the first time to the third time is smaller than the threshold, a process 1300 may be implemented. In some embodiments, the process 1300 may be implemented by the processing device 120 or one or more modules shown in FIG. 2. For example, the process 1300 may be implemented by the determination module 201 and the generation module 203 of the processing device 120. As shown in FIG. 13, the process 1300 may include the following operations.

In 1301, the processing device 120 (e.g., the determination module 201) may obtain first imaging signals collected by an MRI device before radiotherapy rays are emitted.

The operation 1301 may be similar to the operation 1201, and related descriptions may be found in FIG. 12, which are not repeated here.

In 1302, the processing device 120 (e.g., the determination module 201) may determine an initial temporal factor and an initial spatial factor based on first auxiliary signals and the first imaging signals.

The operation 1302 may be similar to the operation 1202. Related descriptions may be found in FIG. 12, which are not repeated here.

In 1303, in response to determining that the second motion amplitude is smaller than or equal to the threshold, the processing device 120 (e.g., the determination module 201) may determine a second updated temporal factor based on third auxiliary signals.

When the second motion amplitude is smaller than or equal to the threshold, it means that the target subject has no obvious motion from the first time to the third time, only the temporal factor needs to be updated, and the initial spatial factor U₀ does not need to be updated. In some embodiments, the determination of the second updated temporal factor may be performed in a similar manner as that of the first updated temporal factor. Detailed descriptions may be found in the operation 1203. For example, the processing device 120 may fill the third auxiliary signals in the K-space to obtain a sixth K-space matrix, and determine the second updated temporal factor by performing the SVD on the sixth K-space matrix. As another example, the processing device 120 may determine the second updated temporal factor based on the sixth K-space matrix and the transformation coefficient T.

In 1304, the processing device 120 (e.g., the generation module 203) may generate the second MRI image of the target subject based on the second updated temporal factor and the initial spatial factor, the second MRI image corresponding to the third time.

The second MRI image may reflect a state of the target subject at the third time. The determination of the second MRI image may be performed in a similar manner as that of the first MRI image. Detailed descriptions may be found in the operation 1204, which are not repeated here.

In some embodiments of the present disclosure, when the target subject moves and returns to the position at the first time, only the temporal factor needs to be updated, and the second MRI image may be generated based on the second updated temporal factor and the initial spatial factor. The second MRI image may be generated with simple calculation, and be output with a low latency, thereby improving the efficiency of the radiotherapy. In some embodiments, the calculation of the basic data can be performed immediately (e.g., before the second imaging phase or simultaneously with the collection of the MRI signals in radiotherapy) after the first auxiliary signals are collected. In this way, the delay in the generation of the second MRI image can be further reduced, thereby further improving the efficiency of the radiotherapy.

FIG. 14 is a flowchart illustrating an exemplary process for generating a third MRI image during MRI-guided radiotherapy according to some embodiments of the present disclosure. In some embodiments, when it is determined that the first motion amplitude from the first time to the second time and the second motion amplitude from the first time to the third time are both greater than the threshold, and the third motion amplitude from the second time to the third time is smaller than the threshold, a process 1400 may be implemented. In some embodiments, the process 1400 may be implemented by the processing device 120 or one or more modules shown in FIG. 2. As shown in FIG. 14, the process 1400 may include the following operations.

In 1401, the processing device 120 (e.g., the acquisition module 202) may obtain first imaging signals collected by an MRI device before radiotherapy rays are emitted.

The operation 1401 may be similar to the operation 1201, and related descriptions may be found in FIG. 12, which are not repeated here.

In 1402, the processing device 120 (e.g., the determination module 201) may determine an initial temporal factor and an initial spatial factor based on first auxiliary signals and the first imaging signals.

The operation 1402 may be similar to the operation 1202. Related descriptions may be found in FIG. 12, which are not repeated here.

When the second motion amplitude is greater than the threshold and the third motion amplitude is smaller than the threshold, it means that the target subject moves and maintains at a new position. To improve the accuracy of imaging, both the temporal factor and the spatial factor need to be updated through operations 1403 and 1404.

In 1403, the processing device 120 (e.g., the determination module 201) may determine a second updated temporal factor based on third auxiliary signals.

Detailed descriptions of the second updated temporal factor may be found in the operation 1303, which are not repeated here.

In 1404, the processing device 120 (e.g., the determination module 201) may determine an updated spatial factor based on second imaging signals collected by the MRI device between the second time and the third time and the second updated temporal factor.

In some embodiments, the determination of the updated spatial factor may be performed in a similar manner as that of the spatial factor U; described in FIG. 6, and related descriptions may be found in FIG. 6.

For example, the updated spatial factor may be determined by solving a seventh optimization function similar to the above Equation (9) or Equation (10). In the seventh optimization function, dₕ denotes K-space data corresponding to the second imaging signals collected by the hth coil channel. Φ₀ may be replaced by the second updated temporal factor. Other coefficients may be the same as those in Equation (9) or Equation (10). In some embodiments, the second imaging signals collected between the second time and the third time may include imaging signals collected at the second time, imaging signals collected at the third time, and imaging signals collected in a time period between the second time and the third time.

As another example, the processing device 120 may update coil sensitivity maps of a plurality of coils for collecting MRI signals based on the second imaging signals. The processing device 120 may further determine the updated spatial factor based on the second updated temporal factor, the second imaging signals, and updated coil sensitivity maps. For example, in the seventh optimization function as aforementioned, Sₕ may denote the updated coil sensitive map corresponding to the hth coil channel. A coil sensitivity map of a coil may reflect a distribution of the response degree of the coil with respect to different portions of the target subject (i.e., the capacity for receiving MRI signals from different portions of the subject). Merely by way of example, for each of the plurality of coils, the processing device 120 may generate a coil image based on the second imaging signals collected by the coil. The processing device 120 may then determine the updated coil sensitivity maps of different coils based on the coil images. In some embodiments, the updated coil sensitivity maps may be determined based on the combination of the first imaging signals and the second imaging signals.

In 1405, the processing device 120 (e.g., the generation module 203) may generate the third MRI image of the target subject based on the second updated temporal factor and the updated spatial factor, the third MRI image corresponding to the third time.

The third MRI image may reflect a state of the target subject at the third time. The generation of the third MRI image may be performed in a similar manner as that of the first MRI image. Detailed descriptions may be found in the operation 1204, which are not repeated here.

According to the process 1400, when the first and second motion amplitudes of the target subject are both greater than the threshold and the third motion amplitude is smaller than the threshold, it means that the target subject maintains at a position at the second time. In this case, the third MRI image of the target subject may be generated based on the second updated temporal factor and the updated spatial factor, which can improve the imaging quality of the third MRI image, thereby improving the accuracy of radiotherapy.

In addition, an obvious motion of a scanned subject (e.g., the motion amplitude is greater than the threshold) may also cause the coil sensitivity maps of the coils to change. According to some embodiments of the present disclosure, if the obvious motion of the scanned subject is detected, the coil sensitivity maps of the coils may be updated, and the value of the updated spatial factor may be determined based on the second imaging signal and the updated coil sensitivity maps. In this way, a more accurate updated spatial factor can be obtained, further improving the imaging quality of the third MRI image, thereby further improving the accuracy of the radiotherapy.

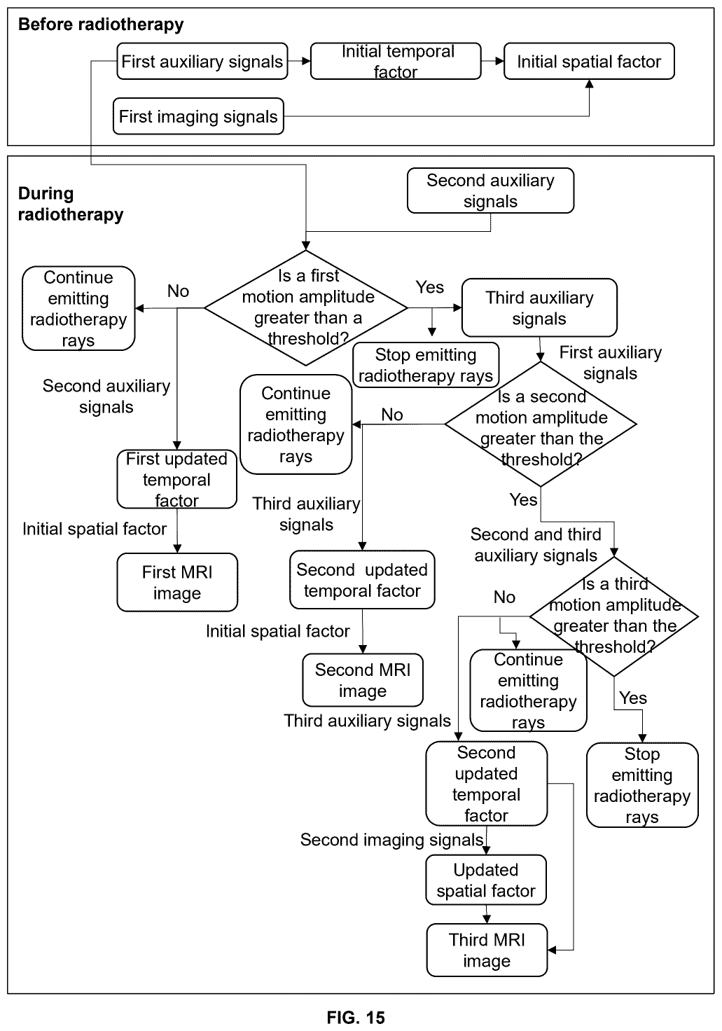

FIG. 15 is a schematic diagram illustrating an exemplary MRI-guided radiotherapy according to some embodiments of the present disclosure. As shown in FIG. 15, an MR device may collect MRI signals before and during radiotherapy. The MRI signals may include auxiliary signals and imaging signals.

The processing device 120 may determine an initial temporal basis and an initial spatial basis based on the MRI signals collected before radiotherapy. Specifically, the processing device 120 may determine the initial temporal factor based on first auxiliary signals collected at a first time before radiotherapy. Further, the processing device 120 may determine the initial spatial factor based on the first auxiliary signals and first imaging signals.

During a radiotherapy process, the processing device 120 may detect a motion of a target subject in real time based on the collected auxiliary signals, and guide the radiotherapy process in real time based on detection results. Specifically, the processing device 120 may obtain second auxiliary signals collected at a second time during the radiotherapy process, and determine whether a first motion amplitude of the target subject from the first time to the second time is greater than a threshold based on the first auxiliary signals and the second auxiliary signals. In response to determining that the first motion amplitude is smaller than or equal to the threshold, the processing device 120 may instruct a radiotherapy device to continue emitting radiotherapy rays. In this case, the processing device 120 may determine a first updated temporal factor based on the second auxiliary signals, and generate a first MRI image corresponding to the second time based on the first updated temporal factor and the initial spatial factor.

In response to determining that the first motion amplitude is greater than the threshold, the processing device 120 may instruct the radiotherapy device to stop emitting the radiotherapy rays and obtain third auxiliary signals collected at a third time, and further determine whether to stop emitting the radiotherapy rays based on the third auxiliary signals. Specifically, the processing device 120 may determine whether a second motion amplitude of the target subject from the first time to the third time is greater than the threshold based on the first auxiliary signals and the third auxiliary signals. In response to determining that the second motion amplitude is smaller than or equal to the threshold, the processing device 120 may instruct the radiotherapy device to continue emitting the radiotherapy rays. In this case, the processing device 120 may determine a second updated temporal factor based on the third auxiliary signals, and generate a second MRI image corresponding to the third time based on the second updated temporal factor and the initial spatial factor.

In response to determining that the second motion amplitude is greater than the threshold, the processing device 120 may determine whether a third motion amplitude of the target subject from the second time to the third time is greater than the threshold based on the second auxiliary signals and the third auxiliary signals. In response to determining that the third motion amplitude is smaller than or equal to the threshold, the processing device 120 may instruct the radiotherapy device to continue emitting the radiotherapy rays. In this case, the processing device 120 may determine a second updated temporal factor based on the third auxiliary signals, and further determine an updated spatial factor based on the second updated temporal factor and the second imaging signals. Then, the processing device 120 may generate a third MRI image corresponding to the third time based on the second updated temporal factor and the updated spatial factor. In response to determining that both the second motion amplitude and the third motion amplitude are greater than the threshold, it means that the target subject does not maintain at a position at the second time, and the processing device 120 may determine to continue to stop emitting the radiotherapy rays.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in smaller than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

What is claimed is:

1. A method for magnetic resonance imaging (MRI)-guided radiotherapy, comprising:
obtaining first auxiliary signals and second auxiliary signals collected by an MRI device during an MRI scan of a target subject, wherein the first auxiliary signals are collected by the MRI device at a first time before radiotherapy rays are emitted, and the second auxiliary signals are collected by the MRI device at a second time after the radiotherapy rays are emitted;
determining, based on the first auxiliary signals and the second auxiliary signals, whether a first motion amplitude of the target subject from the first time to the second time is greater than a threshold; and
determining, based on a determination result of whether the first motion amplitude is greater than the threshold, whether to stop emitting the radiotherapy rays.

2. The method of claim 1, further comprising:
obtaining first imaging signals collected by the MRI device before the radiotherapy rays are emitted;
determining, based on the first auxiliary signals and the first imaging signals, an initial temporal factor and an initial spatial factor;
in response to determining that the first motion amplitude is smaller than or equal to the threshold, determining a first updated temporal factor based on the second auxiliary signals; and
generating, based on the first updated temporal factor and the initial spatial factor, a first MRI image of the target subject, wherein the first MRI image corresponds to the second time.

3. The method of claim 2, wherein the initial temporal factor is determined by:
obtaining a transformation coefficient, the transformation coefficient representing a relationship between auxiliary signals and temporal factors, and
determining, based on the transformation coefficient and the first auxiliary signals, the initial temporal factor.

4. The method of claim 2, wherein the initial temporal factor relates to at least one time-varying dimension of the target subject, the initial spatial factor reflects a relationship between pixel information of the target subject in the image domain and spatial information of the target subject in the physical domain.

5. The method of claim 2, wherein the first MRI image of the target subject is a three-dimension (3D) image.

6. The method of claim 1, further comprising:
in response to determining that the first motion amplitude is greater than the threshold, instructing a radiotherapy device to stop emitting the radiotherapy rays;
obtaining third auxiliary signals collected by the MRI device at a third time, wherein the third time is later than the second time;
determining, based on the first auxiliary signals and the third auxiliary signals, whether a second motion amplitude of the target subject from the first time to the third time is greater than the threshold; and
in response to determining that the second motion amplitude is smaller than or equal to the threshold, instructing the radiotherapy device to continue emitting the radiotherapy rays.

7. The method of claim 6, further comprising:
obtaining first imaging signals collected by the MRI device before the radiotherapy rays are emitted;
determining, based on the first auxiliary signals and the first imaging signals, an initial temporal factor and an initial spatial factor;
in response to determining that the second motion amplitude is smaller than or equal to the threshold, determining a second updated temporal factor based on the third auxiliary signals; and
generating, based on the second updated temporal factor and the initial spatial factor, a second MRI image of the target subject, wherein the second MRI image corresponds to the third time.

8. The method of claim 6, wherein in response to determining that the second motion amplitude is greater than the threshold, the method further comprises:
determining, based on the second auxiliary signals and the third auxiliary signals, whether a third motion amplitude of the target subject from the second time to the third time is greater than the threshold; and
in response to determining that the third motion amplitude is smaller than or equal to the threshold, instructing the radiotherapy device to continue emitting the radiotherapy rays.

9. The method of claim 8, wherein in response to determining that the third motion amplitude is smaller than or equal to the threshold, the method further comprises:
obtaining first imaging signals collected by the MRI device before the radiotherapy rays are emitted;
determining, based on the first auxiliary signals and the first imaging signals, an initial temporal factor and an initial spatial factor;
determining a second updated temporal factor based on the third auxiliary signals;
determining, based on second imaging signals collected by the MRI device between the second time and the third time and the second updated temporal factor, an updated spatial factor; and
generating, based on the second updated temporal factor and the updated spatial factor, a third MRI image of the target subject, wherein the third MRI image corresponds to the third time.

10. The method of claim 9, wherein the determining, based on second imaging signals collected by the MRI device between the second time and the third time and the second updated temporal factor, an updated spatial factor comprises:
determining, based on the second updated temporal factor and the second imaging signals, a reference spatial factor; and
determining the updated spatial factor based on the initial spatial factor and the reference spatial factor.

11. The method of claim 9, wherein the determining, based on second imaging signals collected by the MRI device between the second time and the third time and the second updated temporal factor, an updated spatial factor comprises:
updating, based on the second imaging signals, coil sensitivity maps of coils; and
determining the updated spatial factor based on the second updated temporal factor, the second imaging signals, and the updated coil sensitivity maps.

12. The method of claim 9, wherein the second updated temporal factor is determined based on the third auxiliary signals within a time period of 50 milliseconds.

13. The method of claim 9, wherein the updated spatial factor is determined based on the second imaging signals within a time period of 500 milliseconds.

14. The method of claim 9, wherein the in response to determining that the third motion amplitude is smaller than the threshold, instructing the radiotherapy device to continue emitting the radiotherapy rays includes:
determining, based on the third MRI image, position information of a region of interest (ROI) of the target subject at the third time;
updating, based on the position information, radiation parameters of the radiotherapy rays; and
instructing, based on the updated radiation parameters, the radiotherapy device to continue emitting the radiotherapy rays.

15. The method of claim 1, wherein the first auxiliary signal and the second auxiliary signals have a first readout direction, and
the determining, based on the first auxiliary signals and the second auxiliary signals, whether a first motion amplitude of the target subject from the first time to the second time is greater than a threshold includes:
obtaining first reference auxiliary signals and second reference auxiliary signals collected by the MRI device, wherein the first reference auxiliary signals are collected after the first time, and the second reference auxiliary signals are collected after the second time, the first reference auxiliary signals and the second reference auxiliary signals have a second readout direction, and the second readout direction is different from the first readout direction; and
determining whether the first motion amplitude of the target subject from the first time to the second time is greater than the threshold based on the first auxiliary signals, the second auxiliary signals, the first reference auxiliary signals, and the second reference auxiliary signals.

16. A system for magnetic resonance imaging (MRI)-guided radiotherapy, comprising:
at least one storage device including a set of instructions; and
at least one processor configured to communicate with the at least one storage device, wherein when executing the set of instructions, the at least one processor is configured to direct the system to perform operations including:
obtaining first auxiliary signals and second auxiliary signals collected by an MRI device during an MRI scan of a target subject, wherein the first auxiliary signals are collected by the MRI device at a first time before radiotherapy rays are emitted, and the second auxiliary signals are collected by the MRI device at a second time after the radiotherapy rays are emitted;
determining, based on the first auxiliary signals and the second auxiliary signals, whether a first motion amplitude of the target subject from the first time to the second time is greater than a threshold; and
determining, based on a determination result of whether the first motion amplitude is greater than the threshold, whether to stop emitting the radiotherapy rays.

17. The system of claim 16, further comprising:
obtaining first imaging signals collected by the MRI device before the radiotherapy rays are emitted;
determining, based on the first auxiliary signals and the first imaging signals, an initial temporal factor and an initial spatial factor;
in response to determining that the first motion amplitude is smaller than or equal to the threshold, determining a first updated temporal factor based on the second auxiliary signals; and
generating, based on the first updated temporal factor and the initial spatial factor, a first MRI image of the target subject, wherein the first MRI image corresponds to the second time.

18. The system of claim 17, wherein the initial temporal factor is determined by:
obtaining a transformation coefficient, the transformation coefficient representing a relationship between auxiliary signals and temporal factors, and
determining, based on the transformation coefficient and the first auxiliary signals, the initial temporal factor.

19. The system of claim 17, wherein the initial temporal factor relates to at least one time-varying dimension of the target subject, the initial spatial factor reflects a relationship between pixel information of the target subject in the image domain and spatial information of the target subject in the physical domain.

20. A non-transitory computer readable medium, comprising at least one set of instructions for magnetic resonance imaging (MRI)-guided radiotherapy, wherein when executed by one or more processors of a computing device, the at least one set of instructions causes the computing device to perform a method, the method comprising:
obtaining first auxiliary signals and second auxiliary signals collected by an MRI device during an MRI scan of a target subject, wherein the first auxiliary signals are collected by the MRI device at a first time before radiotherapy rays are emitted, and the second auxiliary signals are collected by the MRI device at a second time after the radiotherapy rays are emitted;
determining, based on the first auxiliary signals and the second auxiliary signals, whether a first motion amplitude of the target subject from the first time to the second time is greater than a threshold; and
determining, based on a determination result of whether the first motion amplitude is greater than the threshold, whether to stop emitting the radiotherapy rays.
